(19) United States
(12) Patent Application Publication
(10) Pub. No.: US 2018/0112200 A1
(43) Pub. Date: Apr. 26, 2018

(54) COMPOSITIONS AND METHODS FOR GENERATING ANTIGENS, ANTIBODIES, AND IMMUNOTHERAPEUTIC COMPOSITIONS AND METHODS

(71) Applicant: The Regents of the University of California, Oakland, CA (US)

(72) Inventors: Hermes J. Garban, Los Angeles, CA (US); Samuel Y. Olson, Los Angeles, CA (US); Kayvan R. Niazi, Agoura Hills, CA (US)

(21) Appl. No.: 15/537,663

(22) PCT Filed: Dec. 21, 2015
(86) PCT No.: PCT/US2015/067053

§ 371 Date: Jun. 19, 2017

Related U.S. Application Data
(60) Provisional application No. 62/095,369, filed on Dec. 22, 2014.

(51) Int. Cl. C12N 9/22; A61K 39/00; C07K 14/47; C07K 14/705

(52) U.S. Cl. CPC C12N 9/22; A61K 39/0011; A61K 2039/64; C07K 14/705; C07K 14/47

(57) ABSTRACT

In some aspects, the invention relates to compositions and methods of generating antigens, wherein the antigen is a biomolecule that is modified by a reactive oxygen species or a reactive nitrogen species. In some aspects, the invention relates to compositions and methods of generating antibodies that bind to biomolecules that have been modified by a reactive oxygen species or a reactive nitrogen species. In some aspects, the invention relates to compositions and methods of generating antibodies that bind to novel epitopes on unmodified biomolecules. In some aspects, the invention relates to the induction of active immunotherapeutic processes (e.g., using preventive or therapeutic vaccines), which may comprise administering neo-antigens generated through methods and compositions described herein.

### RELATED APPLICATIONS

This application claims priority to U.S. Provisional Patent Application No. 62/095,369, filed Dec. 22, 2014, which is incorporated by reference in its entirety.

### BACKGROUND

Oxidative stress, a major component of the immune response, is associated with infection, inflammation, aging, etc. Clinically, a milieu of conditions is associated with oxidative damage including chronic inflammatory and autoimmune diseases, cancer, and age-related disorders. Oxidative stress is mediated in its majority by reactive oxygen species (ROS) and reactive nitrogen species (RNS) among others. ROS are oxygen-based molecules possessing high chemical reactivity. These include biologically-produced free radicals (superoxide and hydroxyl radical, nitric oxide, etc) and non-radical species such as hydrogen peroxide and peroxynitrite.

The exposure of proteins to ROS and RNS alters their composite amino acids and structure thereby generating neo-antigens (a neo-antigen being typically defined as a previously unrecognized host-derived protein which becomes immunogenic after physical/structural or genetic modifications). Oxidative damage to biomolecules, however, is rarely specific and is dependent on the concentration of the protein, its cellular location with respect to cellular oxidant generating systems and the rate of modified protein clearance.

While the direct role of free radicals in causing oxidative damage at the molecular level has been known for decades, the extent to which oxidative damage alters tissue/organ function is still largely elusive. In immunology, oxidative damage has been implicated in several autoimmune diseases, including systemic lupus erythematosus (SLE) where aberrant immune responses against neo-antigens suggest impairment of immune tolerance mechanisms. Factors inducing the formation of neo-antigens include inflammation, infection, drugs, ROS, and environmental factors.

### SUMMARY OF THE DISCLOSURE

In some aspects, the invention relates to compositions and methods of generating antigens, wherein the antigen is a biomolecule that is modified by a reactive oxygen species or a reactive nitrogen species. In some aspects, the invention relates to compositions and methods of generating antibodies that bind to biomolecules that have been modified by a reactive oxygen species or a reactive nitrogen species. In some aspects, the invention relates to compositions and methods of generating antibodies that bind to novel epitopes on unmodified biomolecules. In some aspects, the invention relates to the induction of active immunotherapeutic processes (e.g., using preventive or therapeutic vaccines), which may comprise administering neo-antigens generated through methods and compositions described herein.

### BRIEF DESCRIPTION OF THE FIGURES

FIG. 1. Relevant biological/chemical reactivity of nitric oxide (NO). NO can rapidly react with susceptible chemical moieties in relevant biological proteins: a) e.g., tyrosine residues: nitration (irreversible); b) e.g., thiols: S-nitrosylation (reversible); and c) e.g., transition metal ions: nitrosation (reversible).

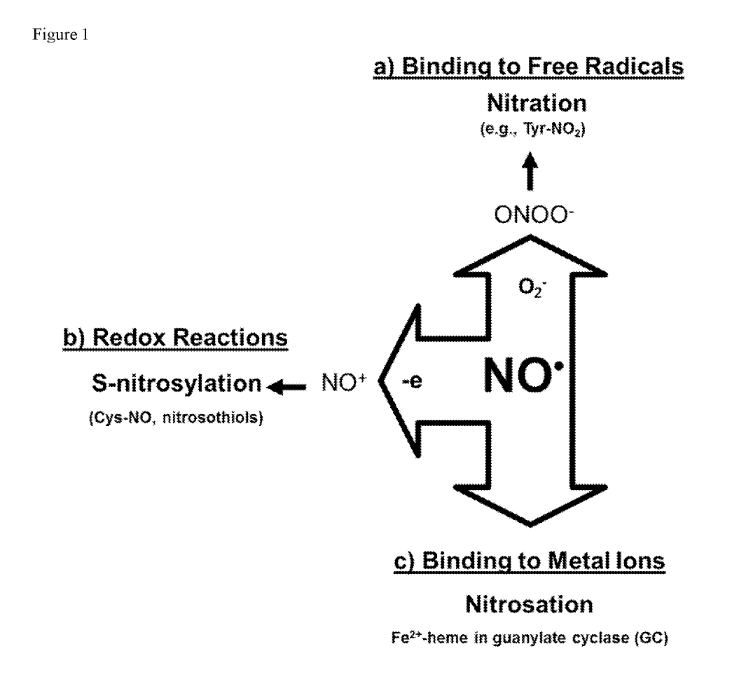

FIG. 2. B16 as a mouse melanoma tumor and immunotherapy model. The subcutaneous model is widely used for the evaluation of therapy in many tumor models, including the poorly immunogenic C57BL/6-derived B16 melanoma. Upon subcutaneous injection, B16 will form a palpable tumor in 5 to 10 days and grow to a minimum of 1×1×1-cm tumor in 14 to 21 days, resulting in increased B16-derived antigen immunogenicity by NO and NO-related molecules: a) Reprogramming: Cultured B16 cells were in vitro-treated to the slow NO-releasing compound DETA-NONOate (250 μM-relatively low concentration) for 18 hours in order to promote the regulation of gene expression resulting in the appearance of new tumor-associated antigens and transforming B16 cells more immunogenic after lysis by sonication and used as antigen (NOVax); b) Modification: Untreated total cultured B16 cell lysate obtained by sonication were incubated in the presence of 31 μM of the NO-derived nitrating agent peroxynitrite (ONOO⁻) at room temperature for 3 hours followed by 48 hours at 4° C. and used as antigen (NiVax). Antigen preparations were frozen and stored at −80° C. until its use in active therapeutic immunizations or for the generation of antiserum for passive therapeutic treatment of tumor-bearing mice.

FIG. 3. Antiserum generation for passive therapeutic treatment (serum transfer) and antibody discovery. A) Non-bearing tumor C57BL/6 female mice (6-12 weeks old) were immunized subcutaneously (SC) with 100 μL (˜100 μg of protein) of either untreated B16 lysate (Control Vax), reprogrammed B16 lysate (NOVax) or modified B16 lysate (NiVax). Boost immunizations with the same dose and concentration of antigen were given at day 7 and 21. Blood was collected 14 days after last booster immunization by cardiac puncture from CO₂-euthanized animals. b) Three doses (20 μL each) of pooled sera from individual experimental group were administered intraperitoneally (IP) to tumor-bearing mice at day 4, 11 and 18 after tumor challenge. Tumor burden was monitored twice weekly.

FIG. 4. Active and passive therapeutic immunization of melanoma. a) Active: B16-F0 tumor-bearing C57BL/6 female mice (6-12 weeks old) were immunized subcutaneously (SC) with 100 μL (˜100 μg of protein) of either untreated B16 lysate (Control Vax or CVax), reprogrammed B16 lysate (NOVax) or modified B16 lysate (NiVax) at day 4, 11 and 18 after tumor challenge. b) Passive: Three doses (20 μL each) of pooled sera from individual experimental group were administered intraperitoneally (IP) to B16-F0 tumor-bearing mice at day 4, 11 and 18 after tumor challenge. Tumor burden was assessed by tumor volume (mm³)±SEM. **=P<0.01|n=10.

FIG. 5. Modified B16 lysate (NiVax)-generated antiserum reacts against non-modified and modified B16 protein lysates. Total protein lysate purified from non-modified B16-F0 (B16), peroxynitrite-modified B16-F0 (NB16) and a non-melanoma mouse cell line EL4 were resolved by SD S-PAGE and immunoblotted using a) control non-immunized antiserum; b) Control untreated B16 lysate (Control Vax) antiserum; c) modified B16 lysate (NiVax) antiserum; and d) no antiserum as primary antibodies. Anti-mouse IgG horse radish peroxidase (HRP)-conjugated was used as secondary antibody.

FIG. 6. Human immunotargets identification. A comprehensive human protein expression microarray (OriGene human protein lysate beta array) was screened for cross-reactivity using modified B16 lysate (NiVax)-derived antiserum as primary antibody and anti-mouse IgG HRP-conjugated was used as secondary antibody.

FIG. 7. Two-dimensional electrophoresis analysis of potential immunotargets. B16-F0 total protein lysate was resolved by two-dimensional (2-D) electrophoresis. Briefly, 2-D analyses of native B16-F0 total protein lysate (˜20 μg) was performed in the first dimension by isoelectric focusing (IEF), using ReadyStrips/Bio-Rad (pH 3-10 nonlinear, 7 cm long). Proteins were separated then on 12% SDS-PAGE and immunoblotted using modified B16 lysate (NiVax)-derived antiserum as primary antibody and anti-mouse IgG HRP-conjugated was used as secondary antibody.

FIG. 8. Two-dimensional electrophoresis analysis of FEN1. B16-F0 total protein lysate was resolved in 2-D electrophoresis and immunoblotted using a polyclonal anti-FEN1 antibody (Cell Signaling).

FIG. 9. Active therapeutic immunization against melanoma using peroxynitrite-nitrated (modified) human FEN1. Purified recombinant human FEN1 protein was modified in the presence of 31 μM and 62 μM of the NO-derived nitrating agent peroxynitrite (ONOO⁻) at room temperature for 3 hours followed by 48 hours at 4° C. and used as antigen for immunization. B16-F0 tumor-bearing mice were immunized subcutaneously (SC) with either saline solution (control) or 100 μL (˜3 μg of protein) of unmodified FEN1 control, 31 μM-modified FEN1 or 62 μM-modified FEN1 at day 4, 11 and 18 after tumor challenge. Tumor burden was assessed by tumor volume (mm³)±SEM. **=P<0.01|n=8.

FIG. 10. Active therapeutic immunization using peroxynitrite-nitrated (modified) human FEN1 prolongs survival. Purified human FEN1 protein was modified in the presence of 31 μM and 62 μM of the NO-derived nitrating agent peroxynitrite (ONOO⁻). B16-F0 tumor-bearing mice were immunized subcutaneously (SC) with either saline solution (control) or 100 μL (˜3 μg of protein) of unmodified FEN1 control, 31 μM-modified FEN1 or 62 μM-modified FEN1 at day 4, 11 and 18 after tumor challenge.

FIG. 11. Passive therapeutic immunization against melanoma using peroxynitrite-nitrated (modified) human FEN1 is mediated by serum antibodies. Antisera generated in non-tumor bearing mice using either unmodified (Control Abs) or modified FEN1 in the presence of 31 μM of peroxynitrite (31 PST Abs) were either antibody-depleted (−) or not (+) using Protein G-coated magnetic beads (Dynabeads® Protein G/Life Technologies). Three doses (20 μL each) of pooled sera from individual experimental group were administered intraperitoneally (IP) to B16-F0 tumor-bearing mice at day 4, 7 and 10 after tumor challenge. Tumor burden was assessed by tumor volume (mm³)±SEM. **=P<0.01|n=8.

FIG. 12. Passive therapeutic immunization against melanoma using modified human FEN1 prolongs survival. Antisera generated in non-tumor bearing mice using either unmodified (Control Abs) or modified FEN1 in the presence of 31 μM of peroxynitrite (31 PST Abs) as previously described were either antibody-depleted (−) or not (+) using Protein G-coated magnetic beads (Dynabeads® Protein G/Life Technologies). Three doses (20 μL each) of pooled sera from individual experimental group were administered intraperitoneally (IP) to B16-F0 tumor-bearing mice at day 4, 7 and 10 after tumor challenge.

FIG. 13. Antibody-dependent antisera immunoreactivity against unmodified B16-F0 total protein lysate. Total protein lysate purified from non-modified B16-F0 was resolved by SDS-PAGE and immunoblotted using: complete control unmodified antiserum (C+), antibody-depleted control unmodified antiserum (C−), complete modified FEN1 antiserum (31+) or, antibody-depleted modified FEN1 antiserum in an independent-lane multiscreening apparatus (Bio-Rad). Anti-mouse IgG HRP-conjugated was used as secondary antibody.

FIG. 14. Novel antigenic determinants discovery. Cultured Leishmania chagasi amastigotes were either lysed and treated in the presence of 31 μM of peroxynitrite at room temperature for 3 hours followed by 48 hours at 4° C. and used as antigen (L-NiVax) to immunize uninfected BALB/c mice (100 μL-SC|˜200 μg/dose) followed by a boost immunization at day 7. Blood was collected 21 days after last booster immunization by cardiac puncture for serum isolation. The same scheme of antiserum preparation was used for antigenic preparations containing saline solution (Vehicle), live L. chagasi amastigotes+Imiquamod (Live L+Imi) and heat-killed L. chagasi amastigotes (Heat-killed L). Total protein lysate from untreated L. chagasi amastigotes was resolved by SDS-PAGE and immunoblotted using: Vehicle, Live L+Imi, Heat-killed L and L-NiVax antisera. Anti-mouse IgG HRP-conjugated was used as secondary antibody.

### DETAILED DESCRIPTION OF THE DISCLOSURE

In some aspects, the invention relates to the finding that the selective induction of oxidative/nitrosative modifications (e.g., nitration) of targeted proteins by specific nitric oxide (NO)-related compounds creates novel disease-altering immunogens by promoting the recognition of novel antigenic determinants. Oxidative/nitrosative damage induced by ROS/RNS may also result in the generation of irreversible, non-denaturating changes in lipids and proteins creating neo-antigens that are highly immunogenic. The oxidation-induced enhancements in immunogenicity observed in autoimmunity support the need for the systematic analysis of other, non-biologically available oxidative/nitration agents in their ability to augment immunity with the goal of creating more effective vaccines, immunotherapeutic means, and diagnostic tools.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. Appropriate methods of administering a substance, a compound or an agent to a subject will also depend, for example, on the age and/or the physical condition of the subject and the chemical and biological properties of the compound or agent (e.g. solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The term “cytotoxic agent” as used herein refers to a substance that inhibits or prevents a cellular function and/or causes cell death or destruction. Cytotoxic agents include, but are not limited to, radioactive isotopes (e.g., At²¹¹, 1¹³¹, 1¹²⁵, Y⁹⁰, Re¹⁸⁶, Re¹⁸⁸, sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “Nitric oxide donor” or “NO donor” refers to compounds that donate, release and/or directly or indirectly transfer a nitrogen monoxide species.

The terms “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono- or di-acid salts can be formed, and such salts may exist in either a hydrated, solvated or substantially anhydrous form. The selection of the appropriate salt will be known to one skilled in the art.

The term “pharmaceutically acceptable basic addition salt” as used herein means any non-toxic organic or inorganic base addition salt of any acid compounds. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition to an asymptomatic subject which reduces the frequency or severity of, or delays the onset of, symptoms of a medical condition in the subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

A “therapeutically effective amount” (“effective amount”) or a “therapeutically effective dose” of a therapy or agent, such as an agonist, antagonist, or inhibitor, is an amount of a drug or therapy that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

### I. Biomolecules, Modified Biomolecules, and Antigens

In some embodiments, the invention relates to an antigen comprising a biomolecule modified by a reactive oxygen species (ROS) or a reactive nitrogen species (RNS). In some embodiments, the invention relates to an antigen comprising a biomolecule modified by a reactive halogen species (RHS). The antigen may be a modified biomolecule. The biomolecule may be a protein, such as a glycoprotein, lipid, or carbohydrate. The biomolecule may be expressed by a cell line, such as a cancer cell line, or pathogen. In some embodiments, the biomolecule is isolated and/or purified. For example, the biomolecule may be a recombinant protein or a synthetic peptide. A modified biomolecule or antigen may be a recombinant protein or a synthetic peptide. The antigen may be a cryptic antigen.

A modified biomolecule or antigen may be a protein or peptide comprising a modified amino acid, such as nitrotyrosine, dinitrotyrosine, chlorotyrosine, dicholortyrosine, dityrosine, 2-amino-3-(3,4-dioxo-cyclohexa-1,5-dienyl)-propionic acid, m-tyrosine, o-tyrosine, L-DOPA (3,4-dihydroxyphenylalanine), nitrophenylalanine, chlorophenylalanine, methionine sulfoxide, methionine sulfone, citrulline (e.g., wherein citrulline replaces arginine), N-γ-nitroarginine, S-nitrocysteine, cysteine sulfenic acid, cysteine sulfinic acid, cysteine sulfonic acid, 2-oxohistidine, asparagine (e.g., wherein asparagine replaces histidine), aspartate (e.g., wherein aspartate replaces histidine), hydroxyproline, pyrrolidone, glutamic semialdehyde, 2-amino-3-ketobutyric acid, α-aminoadipic semialdehyde, hydroxytryptophan, 2-oxo-tryptophan, kynurenine, N-formylkynurenine, hydroxylysine, 2-amino-adipyl-semialdehyde, MDA-lysine, HNE-lysine, acrolein-lysine, carboxymethyllysine, or pHA-lysine. A modified amino acid may be a modified cysteine, methionine, tryptophan, histidine, lysine, or phenylalanine. A modified biomolecule or antigen may comprise S-nitroglutathione. A modified biomolecule or antigen may comprise 4-hydroxynonenal (“HNE”) or malondialdehyde (“MDA”). A modified biomolecule may comprise 13-hydroxy-9Z,11E-octadecadienoic acid, 13-hydroxy-9E,11E-octadecadienoic acid, 9-hydroxy-10E,12-E-octadecadienoic acid (9-EE-HODE), 11-hydroxy-9Z,12-Z-octadecadienoic acid, 4-hydroxynonenal, 13-hydroxy-9Z,11E-octadecadienoic acid, 9-hydroxy-10E,12-Z-octadecadienoic acid, 10-hydroxy-8E,12Z-octadecadienoic acid, 12-hydroxy-9Z-13-E-octadecadienoic, 13-hydroxyoctadecadienoic acid, or 9-hydroxyoctadecadienoic acid.

The biomolecule may be selected from flap structure-specific endonuclease 1 (FEN1); golgi reassembly stacking protein 1 (GORASP1), ArfGAP with GTPase domain-ankyrin repeat and PH domain 1 (AGAP1); microtubule-associated protein tau (MAPT); mitochondrial ribosomal protein L46 (MRPL46); and protocadherin beta 6 (PCDHB6). The biomolecule may be a peptide comprising a subsequence of an amino acid sequence encoding FEN1, GORASP1, AGAP1, MAPT, MRPL46, or PCDHB6. The biomolecule may be a peptide comprising a sequence with at least 95%, 96%, 97%, 98%, or 99% sequence identity with a subsequence of an amino acid sequence encoding FEN1, GORASP1, AGAP1, MAPT, MRPL46, or PCDHB6. A subsequence may be at least 6, 7, 8, 9, 10, 11, 12, 13, 14, 15, 16, 17, 18, 19, 20, 25, 30, 40, 50, 60, 70, 80, 90, or 100 amino acids long.

The biomolecule may be tau, α-synuclein, amyloid (3, or amyloid β precursor protein. The biomolecule may be a peptide comprising a sequence with at least 95%, 96%, 97%, 98%, or 99% sequence identity with a subsequence of an amino acid sequence encoding tau, α-synuclein, amyloid β, or amyloid β precursor protein.

The biomolecule may be abri protein, islet amyloid polypeptide (amylin), a peptide corresponding to exon 1 of huntingtin, prothymosin alpha, the amino-terminal domain of androgen receptor protein, ataxin-1, DRPLA protein (atrophin-1), superoxide dismutase I, beta-2-microglobulin, or calcitonin. The biomolecule may be a peptide comprising a sequence with at least 95%, 96%, 97%, 98%, or 99% sequence identity with a subsequence of an amino acid sequence encoding abri protein, islet amyloid polypeptide (amylin), a peptide corresponding to exon 1 of huntingtin, prothymosin alpha, the amino-terminal domain of androgen receptor protein, ataxin-1, DRPLA protein (atrophin-1), superoxide dismutase I, beta-2-microglobulin, or calcitonin.

The biomolecule may be cystatin c, transthyretin, beta 2 microglobulin, serum amyloid A protein, an imunoglobulin light chain variable domain, insulin, lysozyme (e.g., human lysozyme), alpha lactalbumin, monellin, a ligand- or DNA-binding domain of androgen receptor protein, lactadherein (e.g., medin), gelsolin, apolipoprotein Al, fibrinogen, or atrial natriuretic factor. The biomolecule may be a peptide comprising a sequence with at least 95%, 96%, 97%, 98%, or 99% sequence identity with a subsequence of an amino acid sequence encoding cystatin c, transthyretin, beta 2 microglobulin, serum amyloid A protein, an immunoglobulin light chain variable domain, insulin, lysozyme (e.g., human lysozyme), alpha lactalbumin, monellin, a ligand- or DNA-binding domain of androgen receptor protein, lactadherein (e.g., medin), gelsolin, apolipoprotein Al, fibrinogen, or atrial natriuretic factor.

The biomolecule may be CD52, interleukin 2 receptor, CD30, epidermal growth factor receptor, CD38, interleukin-1β, vascular endothelial growth factor (VEGF), tumor necrosis factor α, tumor necrosis factor β, CD20, cytotoxic T-lymphocyte-associated protein 4 (CTLA-4), CD3, immunoglobulin E, Respiratory Syncytial Virus F protein, receptor tyrosine-protein kinase erbB-2 (HER2/neu), receptor tyrosine-protein kinase erbB-3 (HER3), integrin α4β7, interleukin 12, interleukin 23, interleukin 6 receptor, or integrin α4 subunit.

The biomolecule may be 4-1BB, activin receptor type-2B, activin receptor-like kinase 1, AGS-22M6, alpha-fetoprotein, amyloid α, amyloid β, amyloid precursor protein, angiopoietin-2, anthrax toxin, B-cell activating factor (BAFF), cancer antigen 125 (CA-125/mucin 16), carbonic anhydrase 9 (CA-IX), carcinoembryonic antigen (CEA), C—C chemokine receptor type 4 (CCR4), C—C chemokine receptor type 5 (CCR5), C—C motif chemokine 11 (CCL11), CD2, CD3, CD3ε, CD4, CD6, CD11, CD15, CD18, CD19, CD20, CD22, CD23, CD25, CD28, CD30, CD33, CD37, CD38, CD40, CD40 ligand (CD40L), CD44, CD51, CD52, CD56, CD70, CD74, CD79B, CD80, CD125, CD147, CD152, CD154, CD200, CD221, CD274, CEA-related antigen, chemokine (C—C motif) ligand 2 (CCL2), claudin-18, colony stimulating factor 1 receptor (CSF1R), complement component 5, copper containing amine oxidase 3 (AOC3), cytomegalovirus glycoprotein B, cytotoxic T-lymphocyte-associated protein 4 (CTLA-4), death receptor 5 (DR5/TRAILR2), delta like ligand 4 (DLL4), dipeptidylpeptidase 4, E. coli shiga toxin type-1, E. coli shiga toxin type-2, EGF-like domain-containing protein 7, endosialin, endotoxin, epidermal growth factor receptor (HER1), episialin, epithelial cell adhesion molecule (EpCAM), factor D, fibroblast activation protein alpha, folate receptor 1, Frizzled receptor, glypican 3, granulocyte-macrophage colony-stimulating factor (GM-CSF), growth differentiation factor 8, guanylate cyclase 2C, heat shock protein 90, hepatitis B surface antigen, hepatocyte growth factor/scatter factor (HGF/SF), human scatter factor receptor kinase, huntingtin protein, immunoglobulin E, immunoglobulin epsilon chain C region, Influenza hemagglutinin (HA), insulin-like growth factor 1 (IGF-1) receptor, insulin-like growth factor 2 (IGF-2), integrin α4 subunit, integrin α4β7, integrin α5β1, integrin α7β7, integrin αIIbβ3, integrin αv subunit, integrin αvβ3, integrin β2 subunit, intercellular adhesion molecule 1 (ICAM-1), interferon α, interferon γ, interferon γ-induced protein, interferon α/β receptor, interleukin 1β, interleukin 2 receptor, interleukin 4, interleukin 5, interleukin 6, interleukin 6 receptor, interleukin 9, interleukin 12, interleukin 17, interleukin 17A, interleukin 17F, interleukin 22, interleukin 23, interleukin 31 receptor A, low-density lipoprotein, L-selectin, lymphocyte function-associated antigen 1 (CD11a), lymphotoxin-alpha, lysyl oxidase homolog 2 (LOXL2), macrophage migration inhibitory factor (MMIF), mesothelin, metalloreductase STEAP1, myelin-associated glycoprotein, myostatin, nerve growth factor (NGF), neural apoptosis-regulated proteinase 1, neuropilin-1, NOGO-A, Notch receptor, PD-1, phosphate-sodium co-transporter, platelet-derived growth factor receptor α, platelet-derived growth factor receptor (3, programmed cell death protein 1 (CD279), proprotein convertase subtilisin/kexin type 9 (PCSK9), rabies virus glycoprotein, receptor activator of nuclear factor kappa-B ligand (RANKL), receptor tyrosine-protein kinase erbB-2 (HER2/neu), receptor tyrosine-protein kinase erbB-3 (HER3), Respiratory Syncytial Virus F protein, reticulon-4, Rh blood group D antigen, rhesus factor, sclerostin (SOST), selectin P, SLAM family member 7, syndecan 1, tenascin C, transforming growth factor beta 1 (TGF-β1), transforming growth factor-beta 2 (TGF-β2), transmembrane glycoprotein NMB, trophoblast glycoprotein, tumor necrosis factor α, tumor necrosis factor β, tumor-associated calcium signal transducer 2, tumor-associated glycoprotein 72 (TAG-72), TWEAK receptor, tyrosinase-related protein 1 (TYRP1), vascular endothelial growth factor (VEGF), vascular endothelial growth factor receptor 1, vascular endothelial growth factor receptor 2, or vimentin.

The biomolecule may be a molecule relevant to the pathology of a bacterial infectious disease selected from Anthrax, Bacterial Meningitis, Botulism, Brucellosis, Campylobacteriosis, Cat Scratch Disease, Cholera, Diphtheria, Gonorrhea, Impetigo, Legionellosis, Leprosy (Hansen's Disease), Leptospirosis, Listeriosis, Lyme disease, Melioidosis, MRSA infection, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus (including epidemic typhus), and Urinary Tract Infections.

The biomolecule may be a molecule relevant to the pathology of a parasitic infectious disease selected from Amoebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amoebic infection, Giardiasis, Gnathostomiasis, Hymenolepiasis, Isosporiasis, KaIa-azar, Leishmaniasis, Malaria, Metagonimiasis, Myiasis, Onchocerciasis, Pediculosis, Pinworm Infection, Plasmodium, Scabies, Schistosomiasis, Taeniasis, Toxocariasis, Toxoplasmosis, Trichinellosis, Trichinosis, Trichuriasis, Trichomoniasis, and Trypanosomiasis (including African trypanosomiasis).

The biomolecule may be a molecule relevant to the pathology of a viral infectious disease selected from the group consisting of AIDS, AIDS Related Complex, Chickenpox (Varicella), Common cold, Cytomegalovirus Infection, Colorado tick fever, Dengue fever, Ebola haemorrhagic fever, Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, and Yellow fever. Influenza may be influenza A, B, or C, and influenza A may be subtype H3N2, H1N1, or H5N1. The humoral human immune system recognizes two major immunogenic proteins from the virus, hemagglutinin (HA) and neuraminidase (NA). The known epitope regions of the head or top of the HA molecule correspond to the hypervariable regions. These sequences are highly mutable and isolated sequences have variations within this region. The biomolecule may be hemagglutinin, neuraminidase, or the M2 proton channel (e.g., the M2e peptide) of influenza. The biomolecule may be Glade B gag, protease, reverse transcriptase, gp120, nef peptide, lipopeptide, gp41, or env of HIV. The biomolecule may be envelope glycoprotein (E1/E2) of hepatitis C.

A biomolecule may be a globular protein that undergoes fibrillogenesis and is associated with one or more protein conformational disorders, including cystatin c, transthyretin, beta 2 microglobulin, serum amyloid A protein and its fragments, huntingtin and its fragments (including exon I of huntingtin), immunoglobulin light chain variable domains, insulin, lysozyme (in particular human lysozyme), alpha lactalbumin, monellin, ligand- and DNA-binding domains of androgen receptor protein, lactadherein and more specifically its fragments (e.g., amino acid residues 245-294; medin), gelsolin, apolipoprotein Al, fibrinogen and its fragments, and atrial natriuretic factor.

As specific examples, in Alzheimer's disease, pathology correlates strongly with the presence of a 4 kDa amyloid beta (Aβ) peptide that is part of Aβ peptide precursor (APP), cleaved by enzyme presenilin 1 (PS1). Studies have indicated that the fibrillar form of Aβ1-40 stimulates the microglia and is currently thought to play an important role in the pathogenesis of Alzheimer's disease (Jekabsone, A. et al., J. Neuroinflammation 3:24 (2006)). The peptide sequence of Aβ1-40 is shown in Table 2. On the other hand, Aβ1-42, which is a minor fraction of plaque-forming Aβ, is thought to contribute to the initiation of the formation of fibrillar Aβ. This “long form” of the peptide is described in Table 2. The biomolecule may be, for example, Aβ1-40, Aβ1-42, or a subsequence of either of the foregoing.

A further specific example is Parkinson's Disease (PD). PD is a degenerative neurological disorder affecting 1-2% of the individuals over 50 years of age. The neuropathological hallmarks are characterized by progressive loss of dopaminergic neurons in the substantia nigra pars compacta with the presence of eosinophilic, intracytoplamic, proteinaceous inclusions termed Lewy Bodies (LB). α-Synuclein is the most abundant protein in Lewy Bodies, and it appears to be an important mediator, perhaps even a causal factor, of toxicity in PD. Thus, reduction of toxic α-Synuclein is thought to be beneficial to PD patients. The sequence of one such mouse α-Synuclein peptide, derived from the C-terminal region of the full length protein, is shown in Table 2. Further, elimination or sequestration of nitrated α-Synuclein and fragments thereof appear to have favorable effects on patients suffering from PD. In some embodiments of the invention, the biomolecule is a fragment comprising amino acids 121-137 of human α-Synuclein (DNEAYEMPSEEGYQDYE). In some embodiments, the α-Synuclein fragment (121-137) sequence is substituted at positions 121 and 122 in different species, tri-nitrated at each Y (tyrosine) position, and/or phosphorylated at S129.

In some embodiments, a biomolecule is based on a peptide sequence relevant to prion-diseases. Various species' prion sequences are disclosed by Harmeyer, S. et al., J Gen Virol. 79(Pt 4):937-45 (1998), the entirety of which is hereby incorporated by reference.

In some embodiments, a biomolecule is based on a peptide sequence derived from superoxide dismutase I (SOD1). SOD1 mutation is known to have a causal relationship with the pathology of some forms of familial ALS. It has been reported that the antisera raised against a mutant form of SOD1, human G93A SOD1 recombinant protein, had a protective effect on a mouse model of ALS carrying G37R mutant SOD1, which overexpress human SOD1 protein by 4-fold higher than endogenous mouse SOD1.

Misfolded proteins also play a role in Huntington's disease, a genetic disorder caused by the pathological expansion of a polyglutamine (polyQ) tract in the huntingtin (htt) protein, resulting in neurodegeneration and premature death. A single-chain antibody that binds to an epitope formed by the N-terminal 17 amino acids of huntingtin (Lecerf, J-M et al, Proc Nat'l Acad Sci USA 98(8):4764-4769 (2001)) has been shown to reduce symptoms in a Drosophila model of Huntington's disease. (Wolfgang, W J et al, Proc Nat'l Acad Sci USA. 102(32):11563-11568 (2005)).

A further specific example is Dialysis-related Amyloidosis (DRA). DRA may be caused by different forms of blood filtration, such as haemodialysis, hemofiltration, or Continuous Ambulatory Peritoneal Dialysis (CAPD). DRA has an incidence of greater than 95% in patients on dialysis for more than 15 years with beta-2-microglobulin (B2M) amyloidosis being prevalent and predictably increasing over time. Conformational isomers of B2M have been observed in a clinical setting. B2M is part of the human leukocyte antigen (HLA) class I molecule, and it has a prominent beta-pleated structure characteristic of amyloid fibrils. B2M is known to circulate as an unbound monomer distributed in the extracellular space. B2M undergoes fibrillogenesis to form amyloid deposits in a variety of tissues. This deposition causes renal failure, which causes an increase in synthesis and release of B2M, exacerbating the condition. Thus, in an embodiment of the invention, a biomolecule may be beta 2 microglobulin or a fragment thereof. An exemplary fragment of B2M is the fragment spanning amino acid residues 21-40 in Table 2, useful as a biomolecule of the invention.

The biomolecule may be a peptide comprising a sequence with at least 95%, 96%, 97%, 98%, or 99% sequence identity with a subsequence of an amino acid sequence encoding any one of the foregoing biomolecules. A subsequence may be at least 6, 7, 8, 9, 10, 11, 12, 13, 14, 15, 16, 17, 18, 19, 20, 25, 30, 40, 50, 60, 70, 80, 90, 100, 150, 200, 250, 300, 400, or 500 amino acids long. Peptide sequences with some significance to a disease state or an adverse reaction may be identified through the experimental investigation of a relevant epitope. These sequences may include non-naturally occurring peptide sequences, e.g., that are useful in treating a disease or condition (see, e.g., WO 2006/031727 and U.S. Pat. No. 6,930,168, each of which is hereby incorporated by reference in its entirety).

Further, epitopes may be empirically determined by identifying candidate sequences by positional scanning of synthetic combinatorial peptide libraries, or by making overlapping peptide sequences of the entire protein of interest, and testing those peptides for immune reactivity using, for example, any readout assay useful for such purposes, such as the HI assay, a viral challenge model, or an in vitro or in vivo assay system appropriate for the disease and species for which a novel antigen or antibody is sought. Candidate molecules may include peptides that are modified either during or post synthesis by, for example, sugar- and/or modified sugar addition (such as glycosylation and glycogenation, either N- or S-linked), fatty acid modification (such as myristoylation), or disulfide bond formation. After identifying a candidate epitope, a set of additional related epitopes may be generated using sub-strain variants, cluster variants, drift variants, shift variants, or via modeling and prediction algorithms described in readily available references (e.g., WO 2000/042559, hereby incorporated by reference). Various subsequences that may be used as biomolecules of the invention appear in Tables 1 and 2.

TABLE 1
Subsequences of Viral Proteins that may be used as a
Biomolecule of the Invention

| Peptide | Source/ | Residue |
|---|---|---|
| Sequence | Original Protein | Number |
| ILARNLVPMV | human | 491-500 |
| | cytomegalovirus | |
| | HCMVpp65 | |
| ELEGVWQPA | HCMVpp65 | 526-534 |
| RIFAELEGV | HCMVpp65 | 522-530 |
| NLVPMVATV | HCMVpp65 | 495-503 |
| RIQRGPGRAFVTIGK | HIV-gp120 | V3 loop |
| IIPKSSWSDHEASSGVSSACPYQ | Influenza virus HA | 115-240 |
| GRSSFFRNVVWLIKKDNAYPTIK | protein H5 | |
| RSYNNTNQEDLLVLWGIHHPNDA | Accession Number | |
| AEQTRLYQNPTTYISVGTSTLNQ | ACD62257 | |
| RLVPKIATRSKVNGQSGRMEFF | | |
| WTILKSNDAINK | | |
| CIIPKSSWSDHEASSGVSSACPY | Influenza virus HA | 115-163 |
| QGRSSFFRNVVWLIKKDNAYPTI | protein H5 | flanked by |
| KRSYC | Accession Number | cysteines |
| | ACD62257 | |

TABLE 2
Subsequences of Human Proteins that may be used as
a Biomolecule of the Invention

| | | Source/ | Residue |
|---|---|---|---|
| Relevance | Peptide Sequence | Original Protein | Number |
| Neuro- | DAEFRHDSGYEVHHQKLVFFA | Amyloid beta | 1-40 |
| degeneration | EDVGSNKGAIIGLMVGGVV | | |
| | DAEFRHDSGYEVHHQKLVFFA | Amyloid beta | 1-42 |
| | EDVGSNKGAIIGLMVGGVVIA | | |
| | MGKGEEGYPQEGILEDMPVDP | Mouse alpha | 100-140 |
| | GSEAYEMPSEEGYQDYEEA | synuclein | |
| | DNEAYEMPSEEGYQDYE | Mouse alpha | 121-137 |
| | | synuclein | |
| | MATLEKLMKAFESLKSF | Huntingtin | 1-17 |
| Dialysis- | IQRTPKIQVYSRHPAENGKS | Beta-2 | 21-40 |
| related | | microglobulin | |
| amyloidosis | | | |

A modified biomolecule may comprise an epitope having increased immunogenicity relative to the same epitope on an unmodified biomolecule.

In some embodiments, a biomolecule has been modified by a RNS, and the RNS is nitric oxide or peroxynitrite. The biomolecule may be a protein modified by nitration of a tyrosine, S-nitrosylation of a thiol, or nitrosation of a metal ion. The biomolecule may be modified by nitrosation of a metal ion, wherein the metal ion is iron. The metal ion may be copper, chromium, manganese, or cobalt. In some embodiments, the biomolecule comprises a porphyrin (such as heme, e.g., heme A, B, C, or O) and the biomolecule is modified by nitrosation of a metal ion bound to the porphyrin. The biomolecule may comprise cobalamin, such as methylcobalamin or cobamamide.

A biomolecule may be modified with peroxynitrous acid, peroxynitrite, nitrogen monoxide, nitrogen dioxide, nitrogen dioxide radical, dinitrogen trioxide, nitrosonium cation, nitrosyl sulfate, nitrosyl perchlorate, nitrosonium tetrafluoroborate, nitrosoperoxycarbonate, nitronium cation, a carbonate radical, peroxymonocarbonate, a carboxyl radical, peroxide, hydrogen peroxide, an organic hydroperoxide, a peroxy radical, an alkoxy radical, superoxide, singlet oxygen, a hydroxyl radical, ozone, an oxysulfur radical, a hypohalogen, hypochlorite, hypobromite, hypothiocyanite, nitryl chloride, a halamine, monochloramine, a bromamine, chlorine dioxide, or a phosphate radical.

### II. Antibodies, Antibody Fragments, and Chimeric Antigen Receptors

In some embodiments, the invention relates to an antibody that selectively binds to a modified biomolecule, wherein the affinity of the antibody for the modified biomolecule is greater than the affinity of the antibody for the unmodified biomolecule. For example, the affinity of the antibody for the modified biomolecule might be at least 10%, 20%, 30%, 40%, 50%, 60%, 70%, 80%, 90%, 100%, 200%, 300%, 400%, 500%, 600%, 700%, 800%, 900%, 1000%, 10,000%, or 100,000% higher than the affinity of the antibody for the unmodified biomolecule.

In certain embodiments, the affinity of the antibody for the modified biomolecule is about the same as the affinity of the antibody for the unmodified biomolecule. For example, the affinity of the antibody for the modified biomolecule might be between 0.1× and 10× as much as the affinity of the antibody for the unmodified biomolecule.

In some embodiments, the invention relates to an antibody that selectively binds to an unmodified biomolecule, wherein the affinity of the antibody for the unmodified biomolecule is greater than the affinity of the antibody for the modified biomolecule. For example, the affinity of the antibody for the unmodified biomolecule might be at least 10%, 20%, 30%, 40%, 50%, 60%, 70%, 80%, 90%, 100%, 200%, 300%, 400%, 500%, 600%, 700%, 800%, 900%, 1000%, 10,000%, or 100,000% higher than the affinity of the antibody for the modified biomolecule.

The antibody may be an isolated antibody. In some embodiments, the antibody is a monoclonal antibody. The antibody may be a human, humanized, or chimeric antibody. In some embodiments, the antibody is an IgG1, IgG2, IgG2a, IgG2b, IgG3, or IgG4 antibody.

In certain aspects, the invention relates to an isolated nucleic acid encoding an antibody. In certain aspects, the invention relates to a cell comprising an exogenous nucleic acid encoding an antibody. In certain aspects, the invention relates to a method of producing an antibody comprising culturing a cell that expresses a nucleic acid encoding the antibody.

In certain aspects, the invention relates to a composition comprising the antibody, wherein the antibody is conjugated to a cytotoxic agent.

In certain aspects, the invention relates to an antibody fragment, comprising the antigen-binding region of an antibody. An antibody fragment may be a single-chain variable fragment (scFv). An antibody fragment may be a fragment antigen binding (Fab), chemically linked Fab fragment or Fab fragment including a hinge region F(ab′)₂, dimeric scFv (di-scFv), single-domain antibody (sdAb), trifunctional antibody (3funct), or bi-specific T-cell engager (BiTE).

In certain aspects, the invention relates to a nucleic acid encoding the antibody fragment. In certain aspects, the invention relates to a transformed cell comprising an exogenous nucleic acid encoding the antibody fragment. In certain aspects, the invention relates to methods of producing an antibody fragment, comprising culturing a cell that expresses a nucleic acid encoding the antibody fragment. In certain aspects, the invention relates to a composition comprising the antibody fragment, wherein the antibody fragment is conjugated to a cytotoxic agent.

In some embodiments, the invention relates to a chimeric antigen receptor comprising an antibody or antibody fragment as described herein, such as a scFv. In certain aspects, the invention relates to a nucleic acid encoding the chimeric antigen receptor. In certain aspects, the invention relates to a transformed cell comprising an exogenous nucleic acid encoding the chimeric antigen receptor. In certain aspects, the invention relates to a transformed cell comprising the chimeric antigen receptor. The transformed cell comprising the chimeric antigen receptor may be a lymphocyte, such as a T cell. The transformed cell may be a peripheral blood mononuclear cell. A transformed cell comprising the chimeric antigen receptor may be a human lymphocyte, such as a human T cell.

### III. Cloning Cells, Expression Cells, and Therapeutic Cells

In some aspects, the invention relates to a cell comprising an antibody, antibody fragment, or chimeric antigen receptor as described herein. In some embodiments, a cell comprises a nucleic acid encoding an antibody, antibody fragment, or chimeric antigen receptor as described herein. In some embodiments, a cell expresses an antibody, antibody fragment, or chimeric antigen receptor as described herein.

A cell may be selected from Escherichia coli, Bacillus subtilis, Pseudomonas fluorescens, Leishmania tarentolae, Saccharomyces cerevisiae, Pichia Pastoris, Nicotiana, Drosophila melanogaster, Spodoptera frupperda, Trichoplusia ni, Gallus gallus, Mus musculus, Sus scrofa, Ovis aries, Capra aegagrus, Bos taurus, Sf9 cells, Sf21 cells, Schneider 2 cells, Schneider 3 cells, High Five cells, NS0 cells, Chinese Hamster Ovary (“CHO”) cells, Baby Hamster Kidney cells, COS cells, Vero cells, HeLa cells, and HEK 293 cells. For example, a cell may comprise a nucleic acid encoding an antibody, antibody fragment, of chimeric antigen receptor, and the cell may be E. coli, e.g., for cloning the nucleic acid. A cell may comprise a nucleic acid encoding an antibody or antibody fragment, and the cell may be a CHO cell, e.g., for expressing the antibody or antibody fragment.

A cell may be a lymphocyte, such as a T-cell or a peripheral blood mononuclear cell. A cell may be a lymphocyte and comprise a nucleic acid encoding a chimeric antigen receptor. A cell may be a lymphocyte and comprise a chimeric antigen receptor. A cell may be a lymphocyte and the lymphocyte may express a chimeric antigen receptor. A cell may be a T-cell and the T-cell may express a chimeric antigen receptor, e.g., wherein the chimeric antigen receptor selectively binds to a modified biomolecule. A cell may be modified for allogeneic transplant, e.g., by deleting one or more HLA proteins.

### IV. Vaccines

In certain aspects, the invention relates to a vaccine comprising an antigen. The antigen may be a modified biomolecule as described herein. The vaccine may further comprise a pharmaceutically acceptable carrier.

A vaccine comprising a modified biomolecule may promote active immunization against a target biomolecule that either comprises or does not comprise the modification present in the vaccine. For example, a vaccine comprising a modified biomolecule may induce a subject to generate an auto-immune response against a self-antigen, such as a self-antigen associated with cancer, an inflammatory disease, or a neurodegenerative disease. Similarly, a vaccine comprising a modified biomolecule may induce a subject to generate an immune response against an antigen present on a pathogen or toxin that typically would not elicit an immune response, such as a cryptic epitope of the pathogen. Additionally, a vaccine comprising a modified biomolecule may induce a subject to generate an immune response against a specific epitope of a biomolecule, which may result in a more favorable (e.g., more durable, more intense, or both) immune response than an immune response against an unmodified biomolecule. For example, a modified biomolecule may comprise an epitope of increased immunogenicity relative to the same epitope on an unmodified biomolecule, thereby increasing an immune response against the epitope.

### V. Methods for Producing an Antigen

In certain aspects, the invention relates to a method of producing an antigen, comprising contacting a cell with a reactive oxygen species (ROS) or a reactive nitrogen species (RNS), wherein the ROS or RNS modifies a biomolecule produced by the cell, and the antigen is the modified biomolecule or an epitope on the modified biomolecule. In certain aspects, the invention relates to a method of producing an antigen, comprising contacting a cell with a reactive halogen species (RHS), wherein the RHS modifies a biomolecule produced by the cell, and the antigen is the modified biomolecule or an epitope on the modified biomolecule.

In certain aspects, the invention relates to a method of producing an antigen, comprising contacting a biomolecule with a reactive oxygen species (ROS) or a reactive nitrogen species (RNS), wherein the ROS or RNS modifies a the biomolecule, and the antigen is the modified biomolecule or an epitope on the modified biomolecule. In certain aspects, the invention relates to a method of producing an antigen, comprising contacting a biomolecule with a reactive halogen species (RHS), wherein the RHS modifies the biomolecule, and the antigen is the modified biomolecule or an epitope on the modified biomolecule.

The biomolecule may be a protein or lipid. For example, the biomolecule may be selected from flap structure-specific endonuclease 1 (FEN1); golgi reassembly stacking protein 1 (GORASP1), ArfGAP with GTPase domain-ankyrin repeat and PH domain 1 (AGAP1); microtubule-associated protein tau (MAPT); mitochondrial ribosomal protein L46 (MRPL46); and protocadherin beta 6 (PCDHB6). The biomolecule may be any of the biomolecules described in Section I, supra.

The method may comprise contacting the biomolecule with nitric oxide, a nitric oxide donor (e.g., a NONOate), or a nitrosative agent (e.g., peroxynitrite). For example, the method may comprise incubating the biomolecule with a NONOate compound, wherein the method comprises contacting the biomolecule with nitric oxide, and the NONOate compound produces the nitric oxide. The method may comprise incubating the biomolecule with a NONOate compound, wherein the method comprises contacting the biomolecule with nitric oxide donor, and the NONOate compound is the nitric oxide donor. The NONOate compound may be diethylenetriamine NONOate. The method may comprise contacting the biomolecule with peroxynitrite or any other nitrosative compound.

In some embodiments, a composition comprises the biomolecule, such as a composition comprising a cell (i.e., wherein the cell comprises the biomolecule), a composition comprising a cell lysate, a composition comprising a virus (i.e., wherein the virus comprises the biomolecule), or a composition comprising the biomolecule and a solvent (e.g., water). The method may comprise contacting the composition with nitric oxide, a nitric oxide donor (e.g., a NONOate), or a nitrosative agent (e.g., peroxynitrite). For example, the method may comprise incubating the composition with a NONOate compound, wherein the method comprises contacting the composition with nitric oxide, and the NONOate compound produces the nitric oxide. The method may comprise incubating the composition with a NONOate compound, wherein the method comprises contacting the composition with nitric oxide donor, and the NONOate compound is the nitric oxide donor. The NONOate compound may be diethylenetriamine NONOate. The method may comprise contacting the composition with peroxynitrite or any other nitrosative compound.

A method may comprise contacting a biomolecule with peroxynitrous acid, peroxynitrite, nitrogen monoxide, nitrogen dioxide, nitrogen dioxide radical, dinitrogen trioxide, nitrosonium cation, nitrosyl sulfate, nitrosyl perchlorate, nitrosonium tetrafluoroborate, nitrosoperoxycarbonate, nitronium cation, a carbonate radical, peroxymonocarbonate, a carboxyl radical, peroxide, hydrogen peroxide, an organic hydroperoxide, a peroxy radical, an alkoxy radical, superoxide, singlet oxygen, a hydroxyl radical, ozone, an oxysulfur radical, a hypohalogen, hypochlorite, hypobromite, hypothiocyanite, nitryl chloride, a halamine, monochloramine, a bromamine, chlorine dioxide, or a phosphate radical.

The composition may be incubated with the ROS or RNS for at least 5 minutes, such as at least 10 minutes, at least 20 minutes, at least 30 minutes, at least 45 minutes, at least 1 hour, at least 2 hours, at least 4 hours, at least 8 hours, at least 12 hours, at least 16 hours, or at least 18 hours.

In some embodiments, contacting comprises incubating with a reactive oxygen species, reactive nitrogen species, or reactive halogen species for at least 5 minutes, such as at least 10 minutes, at least 20 minutes, at least 30 minutes, at least 45 minutes, at least 1 hour, at least 2 hours, at least 4 hours, at least 8 hours, at least 12 hours, at least 16 hours, or at least 18 hours.

In some embodiments, an antigen may be produced by synthesizing a biomolecule (e.g., by synthesizing a “modified biomolecule”). For example, an antigen may be a peptide, and producing the antigen may comprise synthesizing the peptide, e.g., using one or more amino acids (or amino acid mixtures) that replicate a feature of oxidative damage. For example, a peptide may be synthesized using nitrotyrosine (i.e., comprising suitable protecting groups, e.g., for FMOC or tBOC chemistry) instead of tyrosine at one or more positions in the amino acid sequence of the peptide. Similarly, a peptide may be synthesized using a mix of nitrotyrosine and tyrosine (e.g., at a ratio of 1:10, 1:5, 1:4, 1:3, 1:2, 1:1) for one or more positions in the amino acid sequence of the peptide.

### VI. Methods for Identifying an Antibody

In some aspects, the invention relates to a method of identifying an antibody, comprising contacting a cell with a reactive oxygen species (ROS) or a reactive nitrogen species (RNS), wherein the ROS or RNS modifies a biomolecule; and selecting an antibody that binds to the modified biomolecule.

In some embodiments, the invention relates to a method of identifying an antibody, comprising contacting a biomolecule with a reactive oxygen species (ROS) or a reactive nitrogen species (RNS) to modify the biomolecule; and selecting an antibody that binds to the modified biomolecule. The biomolecule may be a protein, lipid, or carbohydrate. The biomolecule may be any one of the biomolecules described herein. For example, the biomolecule may be flap structure-specific endonuclease 1 (FEN1); golgi reassembly stacking protein 1 (GORASP1), ArfGAP with GTPase domain-ankyrin repeat and PH domain 1 (AGAP1); microtubule-associated protein tau (MAPT); mitochondrial ribosomal protein L46 (MRPL46); or protocadherin beta 6 (PCDHB6). The biomolecule may be a peptide or polypeptide comprising an amino acid sequence with at least 95%, 96%, 97%, 98%, or 99% sequence homology with a subsequence of an amino acid sequence encoding FEN1, GORASP1, AGAP1, MAPT, MRPL46, or PCDHB6. The biomolecule may be a peptide or polypeptide comprising an amino acid sequence that is a subsequence of an amino acid sequence encoding FEN1, GORASP1, AGAP1, MAPT, MRPL46, or PCDHB6.

The method may comprise contacting the biomolecule with nitric oxide, a nitric oxide donor, or a nitrosative agent. For example, the method may comprise incubating with a NONOate compound under conditions in which the NONOate compound produces nitric oxide. The NONOate compound may be diethylenetriamine NONOate (DETA-NONOate) or diethylamine NONOate (DEA-NONOate). The method may comprise contacting the biomolecule with peroxynitrite. The method may comprise contacting the biomolecule with peroxynitrous acid, peroxynitrite, nitrogen monoxide, nitrogen dioxide, nitrogen dioxide radical, dinitrogen trioxide, nitrosonium cation, nitrosylsulfuric acid, nitrosyl perchlorate, nitrosonium tetrafluoroborate, nitrosoperoxycarbonate, nitronium cation, a carbonate radical, peroxymonocarbonate, a carboxyl radical, peroxide, hydrogen peroxide, an organic hydroperoxide, a peroxy radical, an alkoxy radical, superoxide, singlet oxygen, a hydroxyl radical, ozone, an oxysulfur radical, a hypohalogen, hypochlorite, hypobromite, hypothiocyanite, nitryl chloride, a halamine, monochloramine, a bromamine, chlorine dioxide, or a phosphate radical.

In some embodiments, contacting comprises incubating with a reactive oxygen species, reactive nitrogen species, or reactive halogen species for at least 5 minutes, such as at least 10 minutes, at least 20 minutes, at least 30 minutes, at least 45 minutes, at least 1 hour, at least 2 hours, at least 4 hours, at least 8 hours, at least 12 hours, at least 16 hours, or at least 18 hours.

In some embodiments, contacting comprises incubating with peroxynitrite for at least 5 minutes, such as at least 10 minutes, at least 20 minutes, at least 30 minutes, at least 45 minutes, at least 1 hour, at least 2 hours, at least 4 hours, at least 8 hours, at least 12 hours, at least 16 hours, or at least 18 hours.

Selecting an antibody may comprise exposing an animal to a modified biomolecule and isolating an antibody that the animal produces.

Selecting an antibody may comprise exposing an animal to the modified biomolecule; isolating an antibody-producing cell from the animal; isolating an antibody produced by the cell; and confirming that the antibody binds to the modified biomolecule.

The animal may be a mouse, rat, rabbit, pig, horse, or sheep.

In some embodiments, selecting an antibody comprises selecting an antibody by phage display.

### VII. Methods for Treating Subjects

In some aspects, the invention relates to a method for treating a subject, comprising administering to the subject a composition comprising a modified biomolecule as described herein (active immunization). In other aspects, the invention relates to a method for treating a subject, comprising administering to the subject a composition comprising an antibody or antibody fragment as described herein (passive immunization). In some aspects, the invention relates to a method for treating a subject, comprising administering to the subject a composition comprising a cell as described herein, e.g., wherein the cell comprises a chimeric antigen receptor and/or a nucleic acid encoding a chimeric antigen receptor. In some aspects, the invention relates to a method for treating a subject, comprising administering to the subject a composition comprising a nucleic acid as described herein, e.g., wherein the nucleic acid encodes an antibody, antibody fragment, or chimeric antigen receptor as described herein.

In some aspects, the invention relates to a method for preventing or treating a disease or condition in a subject, comprising administering to the subject a composition comprising a modified biomolecule as described herein (active immunization). In some aspects, the invention relates to a method for preventing or treating a disease or condition in a subject, comprising administering to the subject a composition comprising an antibody or antibody fragment as described herein (passive immunization). In some aspects, the invention relates to a method for preventing or treating a disease or condition in a subject, comprising administering to the subject a composition comprising a cell as described herein, e.g., wherein the cell comprises a chimeric antigen receptor and/or a nucleic acid encoding a chimeric antigen receptor. In some aspects, the invention relates to a method for preventing or treating a disease or condition in a subject, comprising administering to the subject a composition comprising a nucleic acid as described herein, e.g., wherein the nucleic acid encodes an antibody, antibody fragment, or chimeric antigen receptor as described herein.

A subject may be selected from rodents, lagomorphs, felines, canines, porcines, ovines, bovines, equines, and primates. A subject may be a human subject.

A subject may have a neoplasm, and the method may be a method for treating the neoplasm. A subject may have cancer, such as colon cancer, melanoma, ovarian cancer, or breast cancer. A subject may have prostate cancer, stomach cancer, a neuroblastoma, pancreatic cancer, or lung cancer.

A subject may have a viral infection, e.g., and the method may be a method for treating the viral infection. A subject may have a bacterial infection, e.g., and the method may be a method for treating the bacterial infection. A subject may have a parasitic infection (such as leishmaniasis), e.g., and the method may be a method for treating the parasitic infection.

The disease or condition may be a neoplasm. The neoplasm may be cancer. The neoplasm may be a neuroblastoma, glioblastoma, glioma, adenocarcinoma, metastatic brain cancer, adrenocortical carcinoma, sarcoma, ovarian cancer, prostate cancer, breast cancer, lymphoma, Hodgkin's lymphoma, non-Hodgkin's lymphoma, chronic lymphocytic leukemia, acute lymphoblastic leukemia, acute myelogenous leukemia, multiple myeloma, follicular lymphoma, small cell lung cancer, non-small cell lung cancer, gastric cancer, gastrointestinal cancer, colorectal cancer, squamous cell carcinoma, melanoma, head and neck cancer, nasopharyngeal cancer, pancreatic cancer, or renal cell carcinoma.

The disease or condition may be a viral infection, bacterial infection, or a parasitic infection. The disease or condition may be Clostridium difficile, HIV, sepsis, Ebola, leishmaniasis, influenza, Staphylococcus aureus, Candida, Pseudomonas aeruginosa, respiratory syncytial virus, cytomegalovirus, or rabies. A subject may be at risk for developing a Clostridium difficile, HIV, sepsis, Ebola, leishmaniasis, influenza, Staphylococcus aureus, Candida, Pseudomonas aeruginosa, respiratory syncytial virus, cytomegalovirus, or rabies infection. The disease or condition may be AIDS, AIDS Related Complex, Chickenpox (Varicella), Common cold, Cytomegalovirus Infection, Colorado tick fever, Dengue fever, Ebola haemorrhagic fever, Hand, foot and mouth disease, Hepatitis, Herpes simplex, Herpes zoster, Human Papilloma Virus (HPV), Influenza (Flu), Lassa fever, Measles, Marburg haemorrhagic fever, Infectious mononucleosis, Mumps, Poliomyelitis, Progressive multifocal leukencephalopathy, Rubella, SARS, Smallpox (Variola), Viral encephalitis, Viral gastroenteritis, Viral meningitis, Viral pneumonia, West Nile disease, or Yellow fever. A subject may be at risk for developing AIDS, AIDS Related Complex, Chickenpox (Varicella), Common cold, Cytomegalovirus Infection, Colorado tick fever, Dengue fever, Ebola haemorrhagic fever, Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, or Yellow fever. The disease or condition may be Amoebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amoebic infection, Giardiasis, Gnathostomiasis, Hymenolepiasis, Isosporiasis, Kala-azar, Malaria, Metagonimiasis, Myiasis, Onchocerciasis, Pediculosis, Pinworm Infection, Plasmodium, Scabies, Schistosomiasis, Taeniasis, Toxocariasis, Toxoplasmosis, Trichinellosis, Trichinosis, Trichuriasis, Trichomoniasis, or Trypanosomiasis (including African trypanosomiasis). A subject may be at risk for developing Amoebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amoebic infection, Giardiasis, Gnathostomiasis, Hymenolepiasis, Isosporiasis, Kala-azar, Malaria, Metagonimiasis, Myiasis, Onchocerciasis, Pediculosis, Pinworm Infection, Plasmodium, Scabies, Schistosomiasis, Taeniasis, Toxocariasis, Toxoplasmosis, Trichinellosis, Trichinosis, Trichuriasis, Trichomoniasis, or Trypanosomiasis. A subject may have been exposed to a toxin, such as anthrax.

The disease or condition may be an inflammatory disease. The disease or condition may be inflammatory bowel disease, ulcerative colitis, Crohn's disease, rheumatoid arthritis, plaque psoriasis, psoriatic arthritis, ankylosing spondylitis, juvenile idiopathic arthritis, multiple sclerosis, lupus, asthma, systemic scleroderma, dermatomyositis, or polymyositis.

The disease or condition may be a neurodegenerative disease. The disease or condition may be Alzheimer's Disease, Parkinson's Disease, amyotrophic lateral sclerosis, sporadic amyotrophic lateral sclerosis, Lafora disease, or Huntington's disease. The disease or condition may be Dutch hereditary cerebral hemorrhage with amyloidosis (cerebrovascular amyloidosis), congophilic angiopathy, Pick's disease, progressive supranuclear palsy, familial British dementia, Lewy-body related diseases, multiple system atrophy, Hallervorden-Spatz disease, spinocerebellar ataxia, neuronal intranuclear inclusion disease, hereditary dentatorubral-pallidoluysian atrophy, a prion-related disease, scrapie, bovine spongiform encephalopathy, Creutzfeldt-Jakob disease, Gerstmann-Straussler-Scheinker syndrome, kuru, fatal familial insomnia, hereditary cystatin c amyloid angiopathy, or dementia pugilistica.

The disease or condition may be Asperger syndrome, autism, ADHD, hypercholesterolemia, dyslipidemia, atherosclerosis, myocardial infarction, heart failure, ischemia reperfusion injury, ischemic stroke, a thromboembolism, muscular dystrophy, fragile X syndrome, sickle cell disease, paroxysmal nocturnal hemoglobinuria, progeria, lichen planus, vitiligo, bronchopulmonary dysplasia, adult respiratory distress syndrome, emphysema, appendicitis, pancreatitis, acute pancreatitis, alcoholism, diabetes, macular degeneration, uveitis, cataractogenesis, osteoporosis, sarcopenia, chronic fatigue syndrome, or sciatic pain.

A subject may have undergone a transplant, such as an allogeneic transplant or a xenogeneic transplant. A subject may be at risk for organ transplant rejection. A subject may have graft versus host disease.

A subject may have ischemia or a thromboembolism, or the patient may be at risk for ischemia or developing an embolism. A subject may have had a heart attack or an ischemic stroke, or the subject may be at risk for having a heart attack or ischemic stroke.

The disease or condition may be cancer, and the modified biomolecule may be a modified FEN1 protein, or a portion thereof. The method may comprise identifying a subject who has a cancer that overexpresses FEN1, e.g., prior to administering a composition comprising a modified biomolecule, antibody, cell, or nucleic acid to the subject. Cancers that overexpress FEN1 include colon cancer, melanoma, ovarian cancer, breast cancer, prostate cancer, stomach cancer, neuroblastomas, pancreatic cancer, and lung cancer.

The disease or condition may be a neurodegenerative disease, such as Alzheimer's disease or Parkinson's disease, and the modified biomolecule may be a modified tau protein, or a portion thereof. The method may comprise identifying a subject who has a neurodegenerative disease associated with tau. Neurodegenerative diseases that are associated with tau include Alzheimer's disease and Parkinson's disease.

The disease or condition may be leishmaniasis, and the modified biomolecule may be a modified Leishmania protein. The method may comprise identifying a subject who has leishmaniasis. The method may comprise preventing leishmaniasis in a subject, e.g., by prophylactically administering to the subject a modified biomolecule, antibody, or antibody fragment as described herein.

Administering may comprise injecting the composition. Injecting the composition may comprise intravenous injection, subcutaneous injection, intramuscular injection, or intratumoral injection.

This disclosure will be better understood from the Experimental Details which follow. However, one skilled in the art will readily appreciate that the specific methods and results discussed are merely illustrative of the disclosure as described more fully in the embodiments which follow thereafter.

### EXEMPLIFICATION

### Example 1—Method of Generating Antigens and Antibodies Using B16 Cells and Cell Lysates

B16 as a Mouse Melanoma Tumor and Immunotherapy Model.

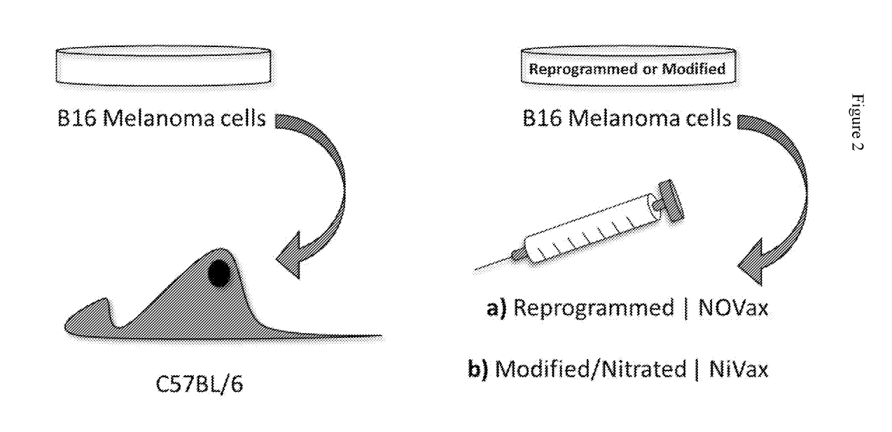

The subcutaneous model is widely used for the evaluation of therapy in many tumor models, including the poorly immunogenic C57BL/6-derived B16 melanoma (FIG. 2). Upon subcutaneous injection, B16 will form a palpable tumor in 5 to 10 days and grow to a minimum of 1×1×1-cm tumor in 14 to 21 days, resulting in increased B16-derived antigen immunogenicity by NO and NO-related molecules. Cultured B16 cells were in vitro-treated to the slow NO-releasing compound Diethylenetriamine NONOate (DETA-NONOate) (250 μM-relatively low concentration) for 18 hours in order to promote the regulation of gene expression resulting in the appearance of new tumor-associated antigens and transforming B16 cells more immunogenic after lysis by sonication and used as antigen (NOVax).

Modification.

Untreated total cultured B16 cell lysate obtained by sonication were incubated in the presence of 31 μM of the NO-derived nitrating agent peroxynitrite (ONOO⁻) at room temperature for 3 hours followed by 48 hours at 4° C. and used as antigen (NiVax). Antigen preparations were frozen and stored at −80° C. until its use in active therapeutic immunizations or for the generation of antiserum for passive therapeutic treatment of tumor-bearing mice.

Antiserum Generation for Passive Therapeutic Treatment (Serum Transfer) and Antibody Discovery.

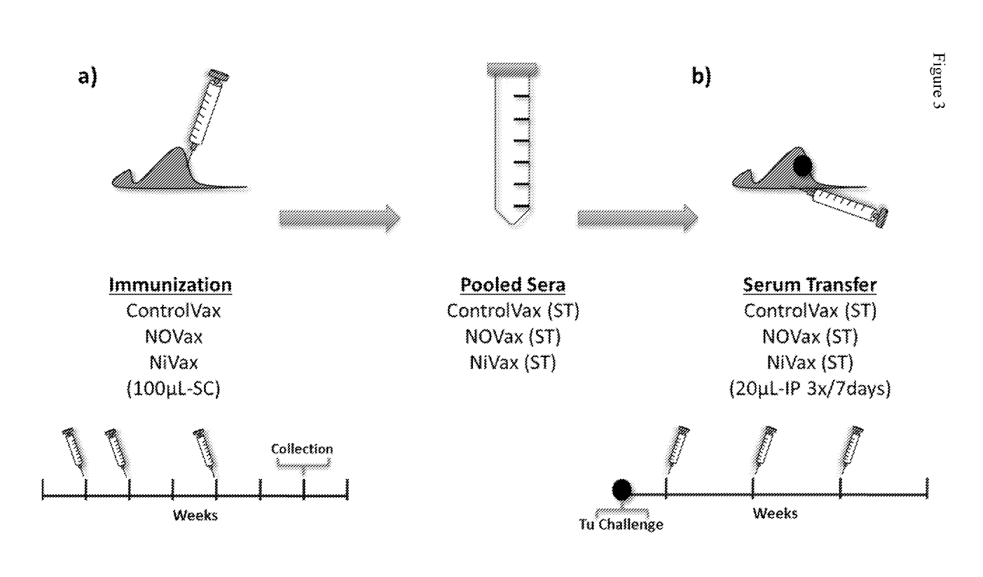

Non-bearing tumor C57BL/6 female mice (6-12 weeks old) were immunized subcutaneously (SC) with 100 μL (˜100 μg of protein) of either untreated B16 lysate (Control Vax), reprogrammed B16 lysate (NOVax) or modified B16 lysate (NiVax). Boost immunizations with the same dose and concentration of antigen were given at day 7 and 21. Blood was collected 14 days after last booster immunization by cardiac puncture from CO₂-euthanized animals. Three doses (20 μL each) of pooled sera from individual experimental group were administered intraperitoneally (IP) to tumor-bearing mice at day 4, 11 and 18 after tumor challenge. Tumor burden was monitored twice weekly. The dosing method is depicted in FIG. 3.

Active Therapeutic Immunization of Melanoma.

B16-F0 tumor-bearing C57BL/6 female mice (6-12 weeks old) were immunized subcutaneously (SC) with 100 μL (˜100 of protein) of either untreated B16 lysate (Control Vax or CVax), reprogrammed B16 lysate (NOVax) or modified B16 lysate (NiVax) at day 4, 11 and 18 after tumor challenge.

Passive Therapeutic Immunization of Melanoma.

Three doses (20 μL each) of pooled sera from individual experimental group were administered intraperitoneally (IP) to B16-F0 tumor-bearing mice at day 4, 11 and 18 after tumor challenge. Tumor burden was assessed by tumor volume (mm³)±SEM. **=P<0.01|n=10.

Results.

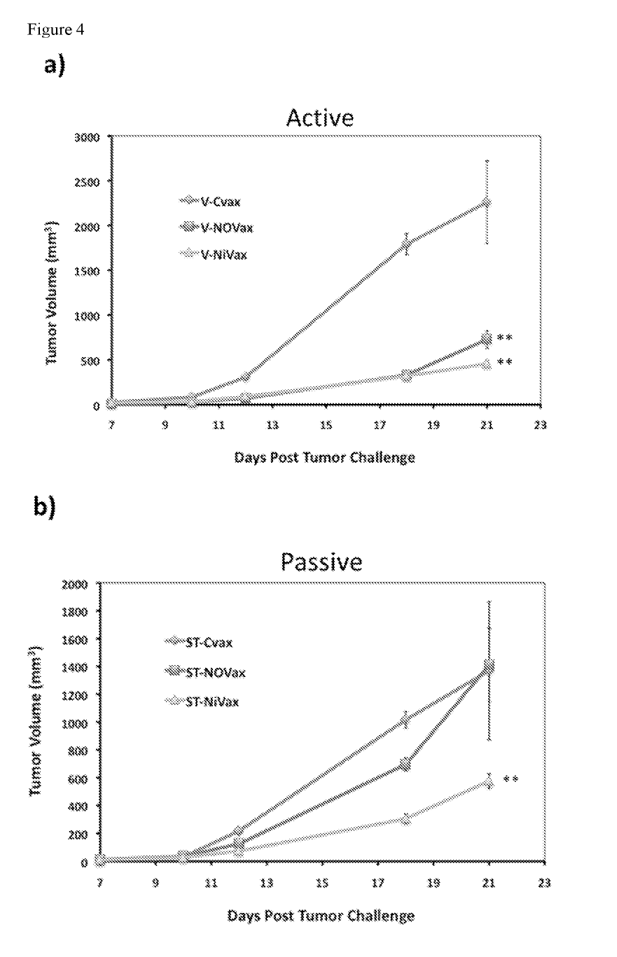

Active immunization with either reprogrammed B16 lysate (NOVax) or modified B16 lysate (NiVax) significantly decrease tumor growth used in a therapeutic manner as compared with untreated B16 lysate (Control Vax or CVax). Only modified B16 lysate (NiVax), however, showed significant tumor growth retardation in a passive serum transfer therapeutic approach, suggesting the presence of tumor inhibitory factors in the serum of modified B16 lysate (NiVax)-immunized mice that are not present in reprogrammed B16 lysate (NOVax)-treated mice (FIG. 4).

### Example 2—Method of Generating Antigens and Antibodies Using B16 Cells and Cell Lysates

Modified B16 Lysate (NiVax)-Generated Antiserum Reacts Against Non-Modified and Modified B16 Protein Lysates.

Total protein lysate purified from non-modified B16-F0 (B16), peroxynitrite-modified B16-F0 (NB16) and a non-melanoma mouse cell line EL4 were resolved by SDS-PAGE and immunoblotted using a) control non-immunized antiserum; b) Control untreated B16 lysate (Control Vax) antiserum; c) modified B16 lysate (NiVax) antiserum; and d) no antiserum as primary antibodies. Anti-mouse IgG horse radish peroxidase(HRP)-conjugated was used as secondary antibody.

Results.

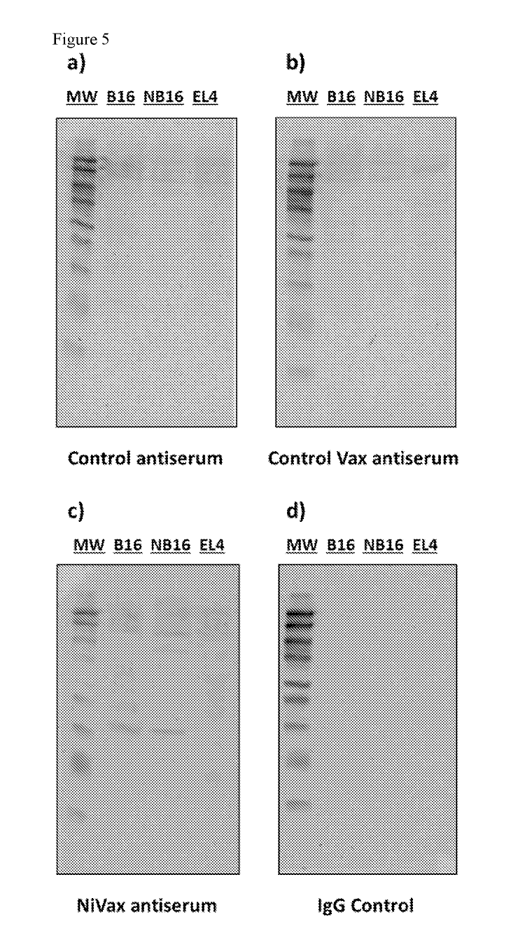

Modified B16 lysate (NiVax) antiserum demonstrated selective immunoreactive activity against modified and non-modified melanoma B16-F0 purified proteins but not against a non-melanoma EL4 (C57BL/6-derived murine thymoma cell line) purified proteins, suggesting the generation of selective immunoreactive antibodies beyond the specific protein modifications (nitration) (FIG. 5).

### Example 3—Identification of Antigens

Human Immunotargets Identification.

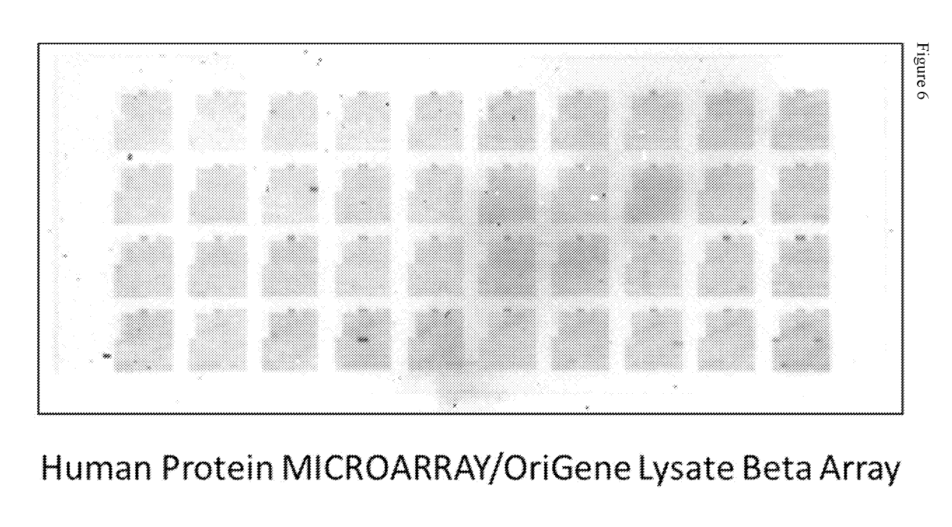

A comprehensive human protein microarray (OriGene human protein lysate beta array) was screened for cross reactivity using modified B16 lysate (NiVax)-derived antiserum as primary antibody and anti-mouse IgG HRP-conjugated was used as secondary antibody (FIG. 6).

Results.

Six novel cross-reactive human immunotargets were identified using the modified B16 lysate (NiVax)-derived antiserum as immunoscreening tool: 1) Flap structure-specific endonuclease 1 (FEN1); 2) Golgi reassembly stacking protein 1 (GORASP1); 3) ArfGAP w/GTPase domain-ankyrin repeat and PH domain 1 (AGAP1); 4) Microtubule-associated protein tau (MAPT); 5) Mitochondrial ribosomal protein L46 (MRPL46); and 6) Protocadherin beta 6 (PCDHB6). These results suggest the potential use of these immunotargets, alone or in combination, as novel melanoma-associated antigens for diagnostic or as immunotherapeutic tools.

### Example 4—Identification of Antigens

Two-Dimensional Electrophoresis Analysis of Potential Immunotargets.

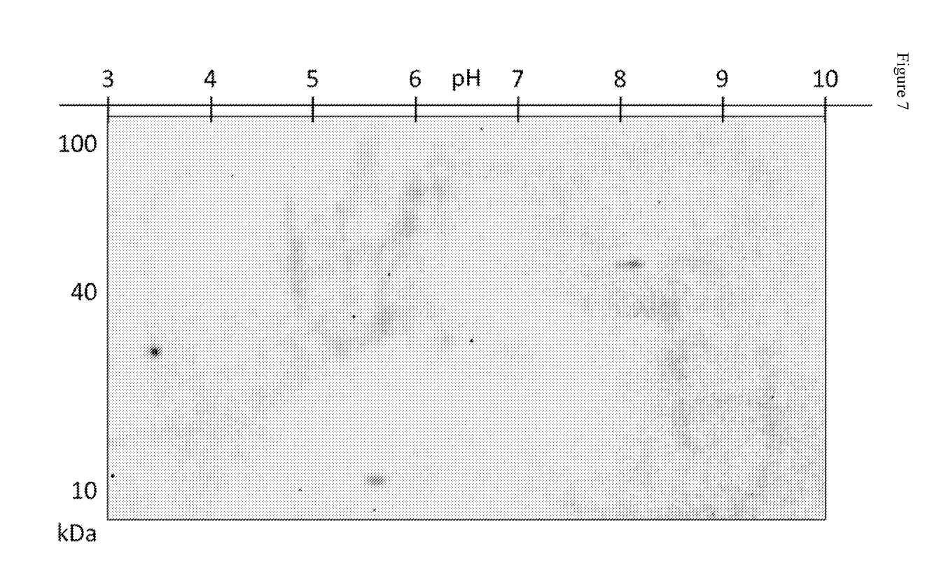

B16-F0 total protein lysate was resolved by two-dimensional (2-D) electrophoresis. Briefly, 2-D analyses of native B16-F0 total protein lysate (˜20 μg) was performed in the first dimension by isoelectric focusing (IEF), using ReadyStrips/Bio-Rad (pH 3-10 nonlinear, 7 cm long). Proteins were separated on 12% SDS-PAGE and immunoblotted using modified B16 lysate (NiVax)-derived antiserum as primary antibody and anti-mouse IgG HRP-conjugated as secondary antibody (FIG. 7). One significant immunoreactive signal was detected coinciding with one of the cross reactive human immunotarget previously identified using the human protein microarray immunoscreening, FEN1 with an IEF around 8 and a molecular weight of approximately 42 kDa.

Two-Dimensional Electrophoresis Analysis of FEN1.

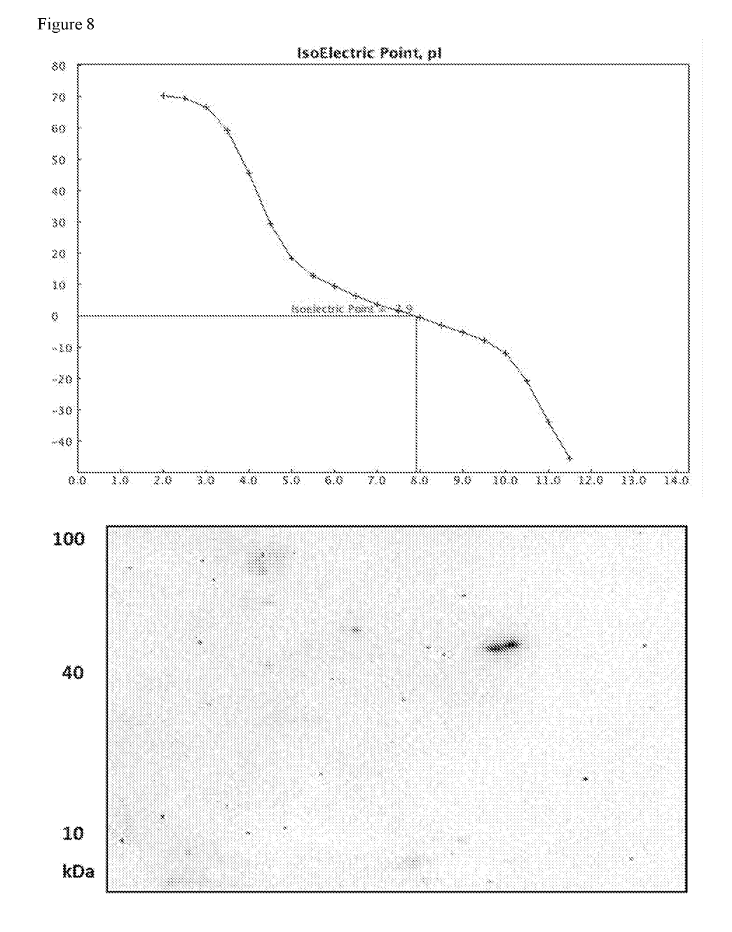

B16-F0 total protein lysate was resolved in 2-D electrophoresis as described above and immunoblotted using a polyclonal anti-FEN1 antibody (Cell Signaling). A specific signal reveled an immunoreactive protein with an IEF of approximately 8 and a molecular weight of approximately 42 kDa, coinciding with one of the significant signals generated by the modified B16 lysate (NiVax)-derived antiserum (FIG. 8). This data suggests that FEN1 may be used for immunotherapy or as a diagnostic tool.

### Example 5—Immunization with Modified FEN1

Active Therapeutic Immunization Against Melanoma Using Peroxynitrite-Nitrated (Modified) Human FEN1.

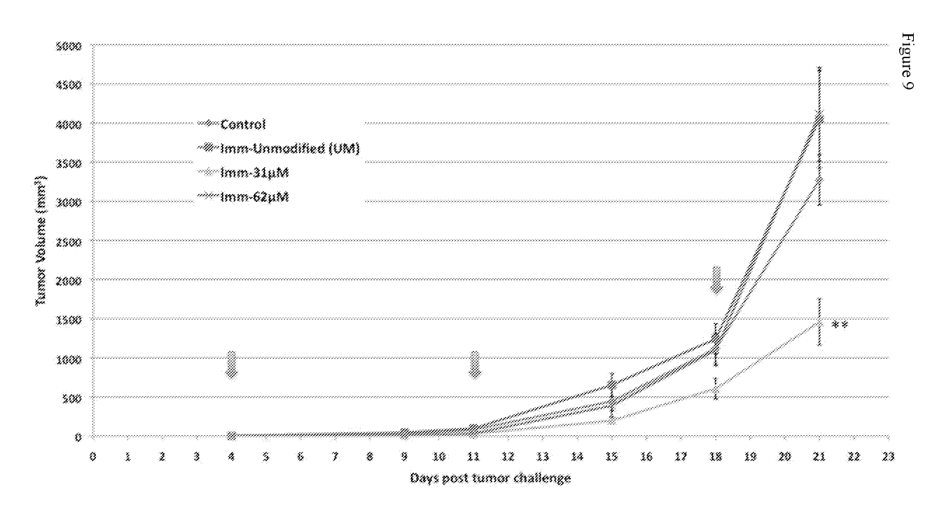

Purified recombinant human FEN1 protein was modified in the presence of 31 μM and 62 μM of the NO-derived nitrating agent peroxynitrite (ONOO⁻) at room temperature for 3 hours followed by 48 hours at 4° C. and used as antigen for immunization. B16-F0 tumor-bearing mice were immunized subcutaneously (SC) with either saline solution (control) or 100 μL (˜3 μg of protein) of unmodified FEN1 control, 31 μM-modified FEN1 or 62 μM-modified FEN1 at day 4, 11 and 18 after tumor challenge. Tumor burden was assessed by tumor volume (mm³)±SEM. **=P<0.01|n=8. Active immunization using 31 μM-modified FEN1 significantly decrease tumor growth used in a therapeutic manner as compared with unmodified FEN1 control or 62 μM-modified FEN1, suggesting an optimal concentration of peroxynitrate at 31 μM for the purified human FEN1 nitration in order to elicit an effective anti-tumor response (FIG. 9).

Active Therapeutic Immunization Using Peroxynitrite-Nitrated (Modified) Human FEN1 Prolongs Survival.

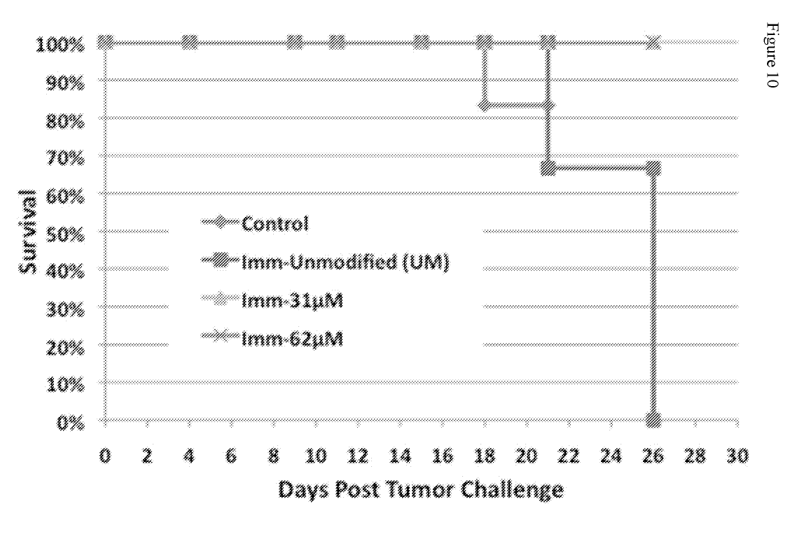

Purified human FEN1 protein was modified in the presence of 31 μM and 62 μM of the NO-derived nitrating agent peroxynitrite (ONOO⁻) as described above. B16-F0 tumor-bearing mice were immunized subcutaneously (SC) with either saline solution (control) or 100 μL (˜3 μg of protein) of unmodified FEN1 control, 31 μM-modified FEN1 or 62 μM-modified FEN1 at day 4, 11 and 18 after tumor challenge. Active immunization using either 31 μM or 62 μM-modified FEN1 significantly increase survival rate of tumor-bearing mice as compared with untreated and unmodified controls (FIG. 10).

### Example 6—the Use of Antibodies Obtained from FEN1-Innoculated Mice

Passive Therapeutic Immunization Against Melanoma Using Peroxynitrite-Nitrated (Modified) Human FEN1 is Mediated by Serum Antibodies.

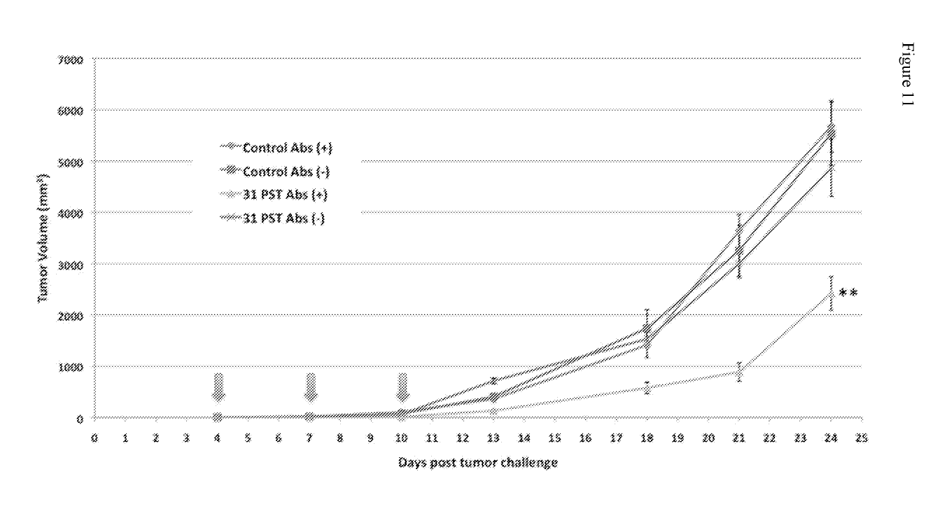

Antisera generated in non-tumor bearing mice using either unmodified (Control Abs) or modified FEN1 in the presence of 31 μM of peroxynitrite (31 PST Abs) as previously described were either antibody-depleted (−) or not (+) using Protein G-coated magnetic beads (Dynabeads® Protein G/Life Technologies). Three doses (20 μL each) of pooled sera from individual experimental group were administered intraperitoneally (IP) to B16-F0 tumor-bearing mice at day 4, 7 and 10 after tumor challenge. Tumor burden was assessed by tumor volume (mm³)±SEM. **=P<0.01|n=8 (FIG. 11). Serum transfer significantly decreased tumor volume. Passive serum transfer of antibody-depleted antiserum from 31 μM-modified FEN1 immunized mice, however, failed controlling tumor growth used in a therapeutic manner as compared with complete serum from 31 μM-modified FEN1 immunized mice, suggesting the specific role of serum containing antibodies in the therapeutic action of passively transferred antiserum from 31 μM-modified FEN1 immunized mice.

Passive Therapeutic Immunization Against Melanoma Using Modified Human FEN1 Prolongs Survival.

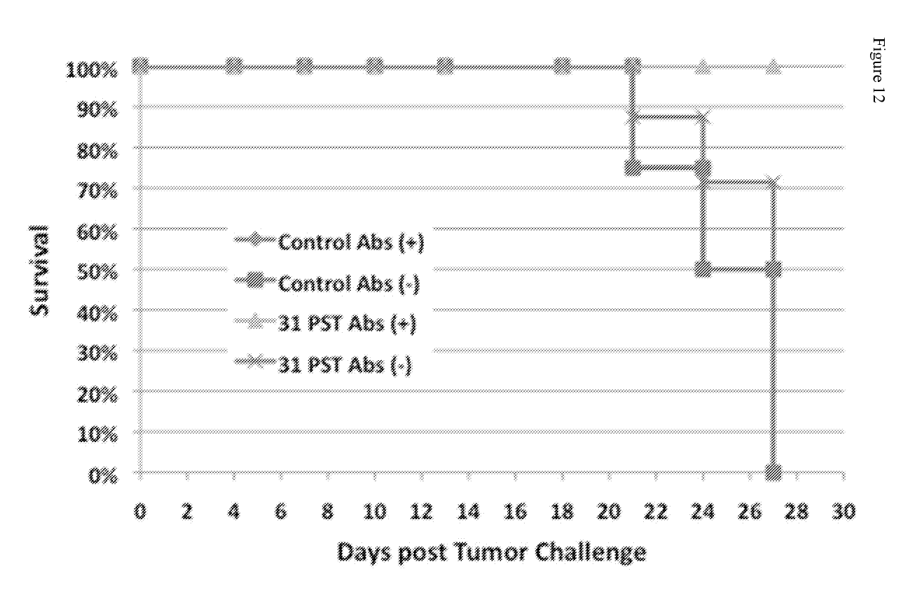

Antisera generated in non-tumor bearing mice using either unmodified (Control Abs) or modified FEN1 in the presence of 31 μM of peroxynitrite (31 PST Abs) as previously described were either antibody-depleted (−) or not (+) using Protein G-coated magnetic beads (Dynabeads® Protein G/Life Technologies). Three doses (20 each) of pooled sera from individual experimental group were administered intraperitoneally (IP) to B16-F0 tumor-bearing mice at day 4, 7 and 10 after tumor challenge (FIG. 12). Serum transfer prolonged survival. Passive serum transfer of antibody-depleted antiserum from 31 μM-modified FEN1 immunized mice, however, failed in maintaining survival after 20 days after tumor challenge when used in a therapeutic manner as compared with complete serum from 31 μM-modified FEN1 immunized mice, suggesting the specific role of serum containing antibodies in the therapeutic action of passively transferred antiserum from 31 μM-modified FEN1 immunized mice.

### Example 7—Antibodies Obtained from FEN1-Innoculated Mice Target Lysates of the B16 Melanoma Cell Line

Antibody-Dependent Antisera Immunoreactivity Against Unmodified B16-F0 Total Protein Lysate.

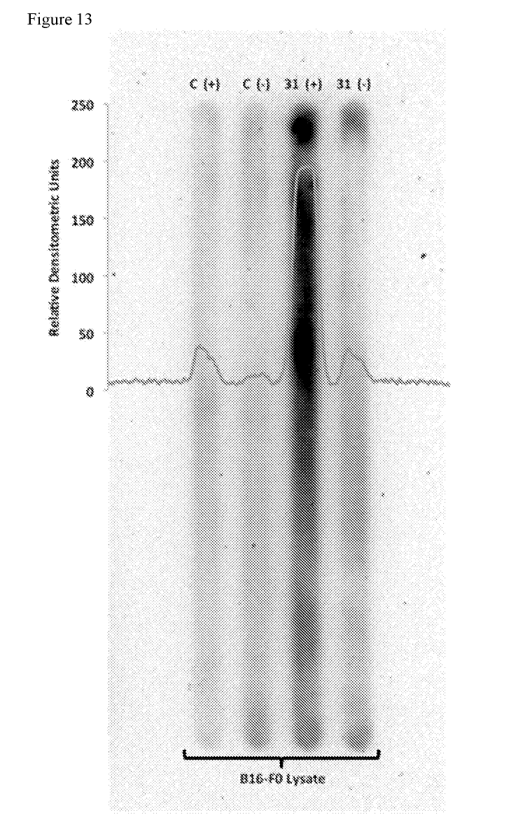

Total protein lysate purified from non-modified B16-F0 was resolved by SDS-PAGE and immunoblotted using: complete control unmodified antiserum (C+), antibody-depleted control unmodified antiserum (C−), complete modified FEN1 antiserum (31+) or, antibody-depleted modified FEN1 antiserum in an independent-lane multiscreening apparatus (Bio-Rad). Anti-mouse IgG HRP-conjugated was used as secondary antibody. Serum obtained from FEN1-innoculated mice reacted specifically against B16-F0 total protein lysate as reflected on the relative densitometric analysis in FIG. 13.

### Example 8—Use of Nitrating Agents to Generate Antibodies that Bind to Novel Epitopes on Unmodified Biomolecules

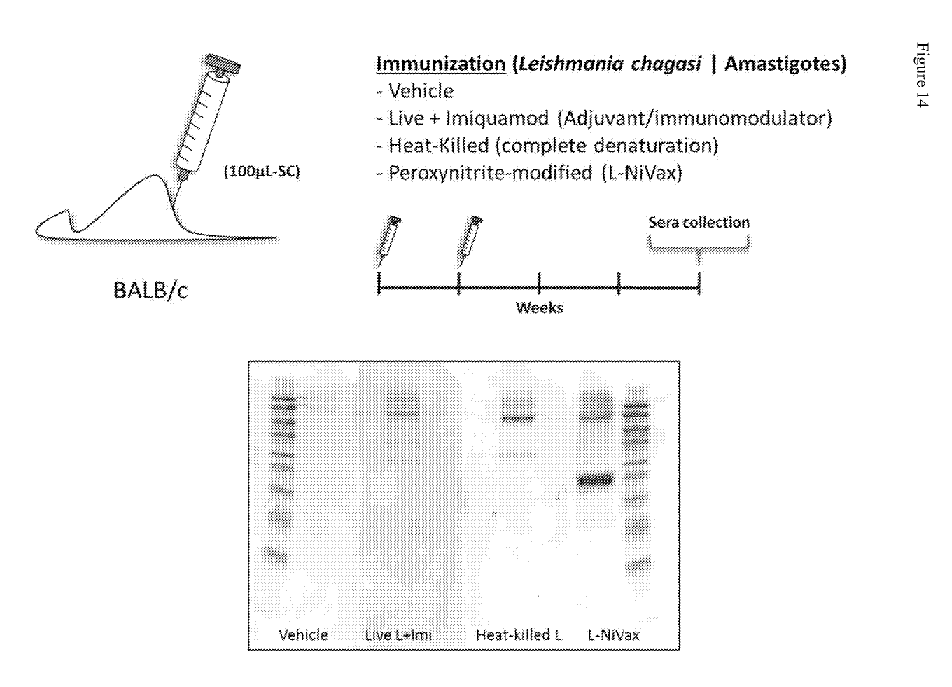

Cultured Leishmania chagasi amastigotes were either lysed and treated in the presence of 31 μM of peroxynitrite at room temperature for 3 hours followed by 48 hours at 4° C. and used as antigen (L-NiVax) to immunize uninfected BALB/c mice (100 μL-SC|˜200 μg/dose) followed by a boost immunization at day 7. Blood was collected 21 days after last booster immunization by cardiac puncture for serum isolation. Same scheme of antiserum preparation was used for antigenic preparations containing saline solution (Vehicle), Live L. chagasi amastigotes+Imiquamod (Live L+Imi) and heat-killed L. chagasi amastigotes (Heat-killed L). Total protein lysate from untreated L. chagasi amastigotes was resolved by SDS-PAGE and immunoblotted using Vehicle, Live L+Imi, Heat-killed L, and L-NiVax antisera. Anti-mouse IgG HRP-conjugated was used as secondary antibody. The Western blot displayed a significant strong immunoreactive band against lysed, untreated L. chagasi amastigotes when using L-NiVax as compared with the other standard modalities of immunization (FIG. 14). These results demonstrate that nitrated antigens can unveil novel, non-nitrated antigenic determinants.

### INCORPORATION BY REFERENCE

All of the patents, published patent applications, and non-patent literature cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An antigen comprising a biomolecule modified by a reactive oxygen species (ROS) or a reactive nitrogen species (RNS).

2-29. (canceled)

30. A method of producing an antigen, comprising contacting a composition comprising a cell with a reactive oxygen species (ROS) or a reactive nitrogen species (RNS), wherein the ROS or RNS modifies a biomolecule produced by the cell, and the antigen is the modified biomolecule.

31. A method of producing an antigen, comprising contacting a composition comprising a biomolecule with a reactive oxygen species (ROS) or a reactive nitrogen species (RNS), wherein the ROS or RNS modifies the biomolecule, and the antigen is the modified biomolecule.

32. The method of claim 30, wherein the biomolecule is a protein or lipid.

33. The method of claim 30, wherein the biomolecule is selected from flap structure-specific endonuclease 1 (FEN1); golgi reassembly stacking protein 1 (GORASP1), ArfGAP with GTPase domain-ankyrin repeat and PH domain 1 (AGAP1); microtubule-associated protein tau (MAPT); mitochondrial ribosomal protein L46 (MRPL46); and protocadherin beta 6 (PCDHB6).

34. The method of claim 31, comprising contacting the composition with nitric oxide, a nitric oxide donor (e.g., a NONOate compound), or a nitrosative agent (e.g., peroxynitrite).

35. The method of claim 34, comprising incubating the composition with a NONOate compound, wherein the method comprises contacting the composition with nitric oxide, and the NONOate compound produces the nitric oxide.

36. The method of claim 34, comprising incubating the composition with a NONOate compound, wherein the method comprises contacting the composition with a nitric oxide donor, and the NONOate compound is the nitric oxide donor.

37. The method of claim 35, wherein the NONOate compound is diethylenetriamine NONOate.

38. (canceled)

39. The method of claim 34, comprising incubating the composition with peroxynitrite, wherein the method comprises contacting the composition with a nitrosative agent, and peroxynitrite is the nitrosative agent.

40-54. (canceled)

55. A method for producing an antigenic biomolecule, comprising contacting a biomolecule with a reactive oxygen species, reactive nitrogen species, or reactive halogen species.

56. The method of claim 55, wherein the reactive oxygen species, reactive nitrogen species, or reactive halogen species is selected from nitric oxide, a nitric oxide donor (e.g., a NONOate), a nitrosative agent, peroxynitrous acid, peroxynitrite, nitrogen dioxide, nitrogen dioxide radical, dinitrogen trioxide, nitrosonium cation, nitrosyl sulfate, nitrosyl perchlorate, nitrosonium tetrafluoroborate, nitrosoperoxycarbonate, nitronium cation, a carbonate radical, peroxymonocarbonate, a carboxyl radical, peroxide, hydrogen peroxide, an organic hydroperoxide, a peroxy radical, an alkoxy radical, superoxide, singlet oxygen, a hydroxyl radical, ozone, an oxysulfur radical, a hypohalogen, hypochlorite, hypobromite, hypothiocyanite, nitryl chloride, a halamine, monochloramine, a bromamine, chlorine dioxide, or a phosphate radical.

57-59. (canceled)

60. The method of claim 55, wherein the biomolecule has at least 95% sequence homology with a subsequence of an amino acid sequence encoding tau, α-synuclein, amyloid β, amyloid β precursor protein, FEN1, GORASP1, AGAP1, MAPT, MRPL46, PCDHB6, 4-1BB, activin receptor type-2B, activin receptor-like kinase 1, AGS-22M6, alpha-fetoprotein, angiopoietin-2, anthrax toxin, B-cell activating factor (BAFF), cancer antigen 125 (CA-125/mucin 16), carbonic anhydrase 9 (CA-IX), carcinoembryonic antigen (CEA), C—C chemokine receptor type 4 (CCR4), C—C chemokine receptor type 5 (CCR5), C—C motif chemokine 11 (CCL11), CD2, CD3, CD3ε, CD4, CD6, CD11, CD15, CD18, CD19, CD20, CD22, CD23, CD25, CD28, CD30, CD33, CD37, CD38, CD40, CD40 ligand (CD40L), CD44, CD51, CD52, CD56, CD70, CD74, CD79B, CD80, CD125, CD147, CD152, CD154, CD200, CD221, CD274, CEA-related antigen, chemokine (C—C motif) ligand 2 (CCL2), claudin-18, colony stimulating factor 1 receptor (CSF1R), complement component 5, copper containing amine oxidase 3 (AOC3), cytomegalovirus glycoprotein B, cytotoxic T-lymphocyte-associated protein 4 (CTLA-4), death receptor 5 (DR5/TRAILR2), delta like ligand 4 (DLL4), dipeptidylpeptidase 4, E. coli shiga toxin type-1, E. coli shiga toxin type-2, EGF-like domain-containing protein 7, endosialin, endotoxin, epidermal growth factor receptor (HER1), episialin, epithelial cell adhesion molecule (EpCAM), factor D, fibroblast activation protein alpha, folate receptor 1, Frizzled receptor, glypican 3, granulocyte-macrophage colony-stimulating factor (GM-CSF), growth differentiation factor 8, guanylate cyclase 2C, heat shock protein 90, hepatitis B surface antigen, hepatocyte growth factor/scatter factor (HGF/SF), human scatter factor receptor kinase, huntingtin protein, immunoglobulin E, immunoglobulin epsilon chain C region, Influenza hemagglutinin (HA), insulin-like growth factor 1 (IGF-1) receptor, insulin-like growth factor 2 (IGF-2), integrin α4 subunit, integrin α4β7, integrin α5β1, integrin α7β7, integrin αIIbβ3, integrin αv subunit, integrin αvβ3, integrin β2 subunit, intercellular adhesion molecule 1 (ICAM-1), interferon α, interferon γ, interferon γ-induced protein, interferon α/β receptor, interleukin 1β, interleukin 2 receptor, interleukin 4, interleukin 5, interleukin 6, interleukin 6 receptor, interleukin 9, interleukin 12, interleukin 17, interleukin 17A, interleukin 17F, interleukin 22, interleukin 23, interleukin 31 receptor A, low-density lipoprotein, L-selectin, lymphocyte function-associated antigen 1 (CD11a), lymphotoxin-alpha, lysyl oxidase homolog 2 (LOXL2), macrophage migration inhibitory factor (MMIF), mesothelin, metalloreductase STEAP1, myelin-associated glycoprotein, myostatin, nerve growth factor (NGF), neural apoptosis-regulated proteinase 1, neuropilin-1, NOGO-A, Notch receptor, PD-1, phosphate-sodium co-transporter, platelet-derived growth factor receptor α, platelet-derived growth factor receptor β, programmed cell death protein 1 (CD279), proprotein convertase subtilisin/kexin type 9 (PCSK9), rabies virus glycoprotein, receptor activator of nuclear factor kappa-B ligand (RANKL), receptor tyrosine-protein kinase erbB-2 (HER2/neu), receptor tyrosine-protein kinase erbB-3 (HER3), Respiratory Syncytial Virus F protein, reticulon-4, Rh blood group D antigen, rhesus factor, sclerostin (SOST), selectin P, SLAM family member 7, syndecan 1, tenascin C, transforming growth factor beta 1 (TGF-β1), transforming growth factor-beta 2 (TGF-β2), transmembrane glycoprotein NMB, trophoblast glycoprotein, tumor necrosis factor α, tumor necrosis factor β, tumor-associated calcium signal transducer 2, tumor-associated glycoprotein 72 (TAG-72), TWEAK receptor, tyrosinase-related protein 1 (TYRP1), vascular endothelial growth factor (VEGF), vascular endothelial growth factor receptor 1, vascular endothelial growth factor receptor 2, or vimentin, wherein the subsequence is at least 6 amino acids long or at least 100 amino acids long.

61-89. (canceled)

90. The method of claim 31, wherein the biomolecule is a protein or lipid.

91. The method of claim 31, wherein the biomolecule is selected from flap structure-specific endonuclease 1 (FEN1); golgi reassembly stacking protein 1 (GORASP1), ArfGAP with GTPase domain-ankyrin repeat and PH domain 1 (AGAP1); microtubule-associated protein tau (MAPT); mitochondrial ribosomal protein L46 (MRPL46); and protocadherin beta 6 (PCDHB6).

92. The method of claim 36, wherein the NONOate compound is diethylenetriamine NONOate.
